(19) United States
(12) Patent Application Publication
(10) Pub. No.: US 2023/0309909 A1
(43) Pub. Date: Oct. 5, 2023

(54) ANALYZING BIOMETRIC SIGNALS TO MONITOR UTERINE CONTRACTIONS

(71) Applicant: Marani Health, Inc., Oakdale, MN (US)

(72) Inventors: Ann Holder, Stillwater, MN (US); Christian Larson, Minneapolis, MN (US); Kathleen Ann Tune, Minneapolis, MN (US); Benjamin Adam Teplitzky, Lakeville, MN (US)

(21) Appl. No.: 18/171,027

(22) Filed: Feb. 17, 2023

Related U.S. Application Data
(60) Provisional application No. 63/268,244, filed on Feb. 18, 2022.

(51) Int. Cl. A61B 5/00; G16H 50/20; A61B 5/391; A61B 5/397

(52) U.S. Cl. CPC A61B 5/4356; G16H 50/20; A61B 5/743; A61B 5/6823; A61B 5/6831; A61B 5/391; A61B 5/7267; A61B 5/397

(57) ABSTRACT

The disclosure describes a system comprising: a memory; and one or more processors in communication with the memory. The one or more processors are configured to: receive, from a set of sensors, biometric data indicative of a muscle contraction of a patient over a period of time; and determine, based on the biometric data, a muscle contraction vector indicating a direction of the muscle contraction over the period of time. Additionally, the one or more processors are configured to determine, based on the biometric data, a likelihood that the muscle contraction comprises a true labor uterine contraction; and output, for display by a user device, the muscle contraction vector indicating the direction of the muscle contraction and the likelihood that the muscle contraction comprises a true labor uterine contraction.

This application claims the benefit of U.S. Provisional Patent Application No. 63/268,244, filed on Feb. 18, 2022, the entire content of which is incorporated herein by reference.

### TECHNICAL FIELD

The disclosure relates generally to medical device systems and, more particularly, medical device systems configured to monitor patient parameters.

### BACKGROUND

Reliable assessment of fetal and maternal well-being is a persistent challenge of current prenatal monitoring technologies, including the non-invasive cardiotocography (CTG) technologies and invasive fetal scalp electrodes. The poor specificity and reliability of these techniques have the potential to lead to adverse maternal and fetal outcomes, including unnecessary cesarean sections, related post-surgical complication, inaccurate detection of fetal hypoxia and other fetal complications.

### SUMMARY

In general, the disclosure describes devices, systems, and/or methods for monitoring maternal and/or fetal physiological parameters during labor and delivery. The maternal and/or fetal data (also referred to herein as “patient data”) may include, for example, one or more fetal and/or maternal biometric signals. In some examples, patient data may include biopotential signals such as maternal and/or fetal electrocardiography (ECG) signals, biometric signals indicating brain activity such as maternal electromyography (EMG) signals, maternal and/or fatal acoustic biometric signals, or any combination thereof. The patient data is not limited to biopotential signals, EMG signals, and acoustic biometric signals. The patient data may include one or more other maternal and/or fetal biometric signals. The patient data may indicate patient health, maternal health, a status of a pregnancy, or any combination thereof.

The patient data may, in some examples, include maternal and/or fetal biometric parameters such as blood pressure, weight, glucose, pH blood levels, blood oxygen level, breathing rate, patient movement, temperature, color spectroscopy, acoustic, and other biometric data. In some examples, one or more biometric parameters of the patient data may be determined using one or more biometric signals of the patient data. For example, a computing system may determine blood pressure using a biopotential signal. In some examples, the patient data may further include data obtained from a mental health assessment, a social determinates of health (SDoH) assessment, socio-economic data for the patient, etc. The patient data may further include any data that may be relevant for the prediction of maternal and/or fetal outcomes, and the disclosure is not limited in this respect.

A computing system may, in some examples, analyze patient data to assist clinicians in monitoring maternal health, fetal health, and pregnancy status. For example, the computing system may identify one or more features or patterns in patient data that indicate patient health, fetal health, pregnancy status, or any combination thereof, and generate an output that supports a clinical team in making health care decisions. The computing system may generate the output to include patient data and analysis of the patient data for display by a user device. Providing patient data and patient data analysis may give a clinician a broader view of maternal health, fetal health, and pregnancy status as compared with systems that provide patient data without patient data analysis. For example, analyzing patient data may support more timely, and more appropriate therapy interventions as compared with systems that do not analyze patient data.

At least some of the patient data may be obtained by a non-invasive wearable device. The wearable device includes a wearable (e.g., a garment or a non-textile element that can be attached to the body) worn about the torso of a pregnant mother and one or more sensors affixed to or embedded in the wearable. The one or more sensors are configured to capture, for example, ECG signals indicative of maternal and/or fetal cardiac activity, EMG signals indicative of uterine contractions, and/or other biopotential signals of the mother or the fetus. The one or more sensors may additionally or alternatively capture signals indicative of one or more other physiological parameters such as blood pressure, weight, glucose, pH blood levels, blood oxygen level, breathing rate, patient movement, temperature, color spectroscopy parameters, acoustic parameters, or any combination thereof.

In some examples, according to one or more techniques of the disclosure, a training data set including patient data and associated outcomes obtained for each of a plurality of patients (e.g., pregnant human mothers and their fetuses) is used to train one or more machine learning models for maternal and/or fetal outcome prediction. The machine learning model(s) relate various features of the patient data to monitor maternal health, fetal health, pregnancy status, or any combination thereof. In some examples, according to one or more techniques of the disclosure, a cloud-based pregnancy monitoring system receives patient data associated with a pregnant mother, applies the patient data to the trained machine learning model, and predicts one or more fetal outcomes, maternal outcomes, and or pregnancy status based on the patient data.

A computing system may, in some examples, include the wearable device configured to collect one or more biometric signals during pregnancy and a computing device configured to monitor the one or more biometric signals during pregnancy, labor, and delivery. The computing system may provide data in real-time, allowing users to view the data, provide feedback to the patient, and/or make healthcare decisions. For example, the real-time data may indicate a need for one or more emergency procedures and provide information that allows clinicians to provide advice during delivery. In some examples, the patient may view the real-time data and make decisions based on this data. The wearable device may include a variety of sensors such that if one or more sensors loses a signal indicating a parameter, the system may adjust to using one or more other sensors to measure the same parameter.

The computing system may, in some examples, analyze patient data in order to monitor a pregnancy status of the patient. For example, the computing system may identify one or more uterine contractions. Electrical biopotential signals (e.g., ECG signals) collected by the wearable device may, in some examples, indicate one or more uterine contractions. Uterine contractions include true labor uterine contractions, which occur during labor. Uterine contractions also include contractions that do not necessarily occur during labor and are separate from true labor uterine contractions. Braxton-Hicks contractions, for example, represent non-labor uterine contractions that start as early as six weeks into pregnancy. The computing system may, in some examples, analyze patient data to identify uterine contractions and determine a likelihood that uterine contractions represent true labor uterine contractions. This may assist clinicians in determining a status of a pregnancy, such as whether or not a mother is in labor.

The techniques of this disclosure may provide one or more advantages. For example, the computing system may analyze patient data to determine a likelihood that one or more uterine contractions represent true labor uterine contractions. The computing system may additionally or alternatively generate a muscle contraction vector indicating a direction of a muscle contraction over a period of time. Since true labor uterine contractions may have a direction relative to the body of the patient that is different than a direction of one or more non-labor uterine contractions, the direction of a uterine contraction may indicate a likelihood of whether the contraction is a true labor uterine contraction. The computing system may, in some examples, output both the determined likelihood and the muscle contraction vector for display by a user device. Additionally, or alternatively, the system may output patient data for display by the user device. This may provide clinicians with more information to make medical decisions as compared with systems that do not output a determined likelihood, a muscle contraction vector, and/or patient data.

In some examples, a system comprises a memory; and one or more processors in communication with the memory, wherein the one or more processors are configured to: receive, from a set of sensors, biometric data indicative of a muscle contraction of a patient over a period of time; determine, based on the biometric data, a muscle contraction vector indicating a direction of the muscle contraction over the period of time; determine, based on the biometric data, a likelihood that the muscle contraction comprises a true labor uterine contraction; and output, for display by a user device, the muscle contraction vector indicating the direction of the muscle contraction and the likelihood that the muscle contraction comprises a true labor uterine contraction

In some examples, a method includes receiving, by one or more processors from a set of sensors, biometric data indicative of a muscle contraction of a patient over a period of time, wherein the one or more processors are in communication with a memory; determining, by the one or more processors based on the biometric data, a muscle contraction vector indicating a direction of the muscle contraction over the period of time; determining, by the one or more processors based on the biometric data, a likelihood that the muscle contraction comprises a true labor uterine contraction; and outputting, by the one or more processors for display by a user device, the muscle contraction vector indicating the direction of the muscle contraction and the likelihood that the muscle contraction comprises a true labor uterine contraction.

In some examples, a computer readable medium includes instructions that when executed cause one or more processors to: receive, from a set of sensors, biometric data indicative of a muscle contraction of a patient over a period of time; determine, based on the biometric data, a muscle contraction vector indicating a direction of the muscle contraction over the period of time; determine, based on the biometric data, a likelihood that the muscle contraction comprises a true labor uterine contraction; and output, for display by a user device, the muscle contraction vector indicating the direction of the muscle contraction and the likelihood that the muscle contraction comprises a true labor uterine contraction.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF DRAWINGS

FIG. 1A is a diagram of an example system for the acquisition and communication of patient data and prediction of maternal and/or fetal outcomes using trained machine learning model(s) in accordance with one or more techniques of the disclosure.

FIG. 1B is a diagram of an example system for the acquisition and communication of patient data during labor and delivery, in accordance with one or more techniques of the disclosure.

FIG. 2 is a more detailed block diagram of the electronic components of an example wearable device and an example patient computing device in accordance with one or more techniques of the disclosure.

FIG. 3 is a block diagram of an example fetal monitoring system (FMS) in accordance with one or more techniques of the disclosure.

FIG. 4A is a diagram of a first example wearable device including a first one or more sensors embedded or affixed to a wearable garment, in accordance with one or more techniques of the disclosure.

FIG. 4B is a diagram of a second example wearable device including a first one or more sensors embedded or affixed to a wearable element and a second one or more sensors embedded or affixed to a wearable element, in accordance with one or more techniques of the disclosure.

FIG. 4C is a diagram of a third example wearable device including a first one or more sensors embedded or affixed to a wearable element and a second one or more sensors, in accordance with one or more techniques of the disclosure.

FIG. 4D is a conceptual diagram illustrating a cross-section view of the abdomen of the patient and the wearable element, in accordance with one or more techniques of this disclosure.

FIG. 4E is a block diagram illustrating electronic components of an example wearable device, in accordance with one or more techniques of this disclosure.

FIGS. 5A-5D are graphs illustrating an example mixed maternal-fetal ECG signal, a fetal ECG signal extracted from the mixed maternal-fetal ECG signal, a graph showing an identification of peaks in the fetal heart rate, and a presentation of fetal heart rate as determined based on the fetal ECG signal, respectively, in accordance with one or more techniques of the disclosure.

FIGS. 6A-6D are graphs showing an example abdominal signal obtained using the example wearable device, the envelope of the original uterine activity (UA) and smoothed UA signal, the detected uterine activity signal, and an example combined fetal heart rate and uterine signal graph, respectively, in accordance with one or more techniques of the disclosure.

FIG. 7 is a conceptual diagram illustrating an example of training and using a machine learning model that predicts one or more outcomes and/or generates uterine contraction information, in accordance with one or more techniques of the disclosure.

FIG. 8 shows an example visualization of a convolutional neural network (CNN) for classification (e.g., prediction) using ECG signal data.

FIG. 9 shows an example segmentation batch using a thumbing window.

FIG. 10 is a flow chart illustrating an example process by which a computing device may train one or more machine learning models to generate one or more maternal and/or fetal outcome predictions in accordance with one or more techniques of the disclosure.

FIG. 11 is a flow chart illustrating an example process by which a computing device may predict one or more maternal and/or fetal outcomes in accordance with one or more techniques of the disclosure.

FIG. 12 is a flow chart illustrating an example process by which a computing device may generate one or more maternal and/or fetal outcome predictions based on longitudinal tracking of a particular patient in accordance with one or more techniques of the disclosure.

FIG. 13 is a flow diagram illustrating an example operation for generating information corresponding to one or more uterine contractions of a patient, in accordance with one or more techniques of this disclosure.

### DETAILED DESCRIPTION

In general, the disclosure describes devices, systems, and/or methods for predicting maternal and/or fetal health outcomes based on maternal and/or fetal data. The maternal and/or fetal data (also referred to herein as “patient data”) may include, for example, data regarding sensed biopotential signals such as maternal and/or fetal electrocardiography (ECG) signals, maternal electromyography (EMG) signals, and/or other biopotential signals. The patient data may further include maternal and/or fetal biometric data such as blood pressure, weight, glucose, pH blood levels, blood oxygen level, breathing rate, patient movement, temperature, and other biometric data. In some examples, the patient data may further include data obtained from a mental health assessment, a social determinates of health (SDoH) assessment, socio-economic data, etc. The patient data may further include any data that may be relevant for the prediction of maternal and/or fetal outcomes, and the disclosure is not limited in this respect.

The techniques may assist clinicians in identification of features or patterns in patient data that could lead to sub-optimal outcomes, support real time decision-making by the clinical team, thus helping to promote timely, appropriate interventions, and decrease overall costs associated with adverse maternal and fetal outcomes. The techniques may aid clinicians and healthcare providers to improve prenatal care and to better manage risk pregnancy patients while at home, allowing for continued monitoring and alert triggering. In addition, healthcare costs associated with pregnancy may be reduced by eliminating unnecessary travels and clinic visits, saving time and stress to future mothers. In addition, collection of relevant patient data may provide a framework for clinical and scientific research in the field of prenatal care and support continuous updates and refinements to the predictive models and the resulting predicted maternal and/or fetal outcomes.

In some examples, according to one or more techniques of the disclosure, a training data set including patient data and associated outcomes obtained for each of a plurality of patients (e.g., pregnant human mothers and their fetuses) is used to train a machine learning model for maternal and/or fetal outcome prediction. The machine learning model is indicative of features of the patient data are predictive of one or more maternal or fetal outcomes (either adverse or non-adverse).

In some examples, according to one or more techniques of the disclosure, a cloud-based pregnancy monitoring system receives patient data associated with a pregnant mother, applies the patient data to the trained machine learning model, and predicts one or more fetal and/or maternal outcomes associated with the pregnant mother based on the patient data.

Although specific examples using maternal and/or fetal ECG or heart rate data to predict one or more outcomes are described herein, it shall be understood that the disclosure also applies to prediction of outcomes using any other type of patient data, including other sensed biopotential signals, biometric data, socio-economic data, mental health data or any other data relevant to prediction of maternal and/or fetal outcomes, and that the disclosure is not limited in this respect.

The techniques of the disclosure may predict and output one or more maternal and/or fetal outcomes. Predicted fetal outcomes may include, but are not limited to, Apgar scores (e.g., 1, 5 and 10 minutes after birth), cord blood gas pH level, neonatal destination immediately after birth, admission to Neonatal Intensive Care Unit (NICU) within 48 hours of birth, NICU length of stay, resuscitation intervention, other neonatal complications, neonatal death up to 28 days after birth, etc. Predicted maternal outcomes may include, but are not limited to, mode of delivery (e.g., vaginal or C-section), reason for C-section, grade of C-section (If performed—Grades 1, 2, 3 or 4), length of stay, destination immediately after birth, admission to a higher level of care, complications (type and severity), hour of day of delivery, day of week of delivery, etc.

In some examples, one or more techniques of the disclosure combine patient data such as maternal and/or fetal ECG or heart rate data with additional patient data including biometric data such as uterine contraction data, Braxton-Hicks contraction data, blood pressure, weight, glucose, pH blood levels, blood oxygen level, breathing rate, patient movement, temperature; patient health assessment data such as results of a mental health assessments, a social determinates of health (SDoH) assessment, data regarding preexisting conditions, patient usage patterns (for example, the timing or update patterns when answering questions on a psychological survey), time of day, frequency or time between measurements, and/or any other patient data relevant to prediction of maternal and/or fetal outcomes for use as training data and/or input data for a current monitoring session for which one or more outcomes are predicted.

The training data may be used to generate one or more ML models for the identification of high-risk pregnancies (e.g., prediction of one or more adverse outcomes described herein). The techniques of the disclosure may help identify false predictions of fetal distress that may lead to unnecessary Cesarean sections, so that unnecessary C-Sections and the associated increase in health care costs and maternal recovery time may be minimized. At the same time, accuracy regarding the prediction of actual fetal distress may be maximized, allowing for timely interventions when needed. The techniques of the disclosure thus provide a comprehensive and accurate monitoring system that takes many types, attributes, features, and/or patterns of fetal and/or maternal data into account when predicting one or more maternal and/or fetal outcomes.

In some examples, the techniques of the disclosure include a wearable device for acquiring maternal and/or fetal biopotential (such as ECG and/or EMG) or heart rate data that a pregnant mother can use at home or other non-clinical environment, which in combination with a cloud-based remote monitoring system (e.g., telehealth and/or telemedicine system), may improve the mother's comfort and peace of mind during pregnancy. The techniques may be used to monitor the health of prenatal and postpartum patients in a remote monitoring setting. The techniques of the disclosure may also be used during labor and delivery in addition to or instead of a traditional cardiotocography (CTG) monitoring device in clinical/hospital environment.

In some examples, one or more techniques of this disclosure include sensing one or more physiological parameters in real-time during pregnancy. In some examples, one or more techniques of this disclosure include sensing one or more parameters in real-time for display to a clinician during labor and/or delivery. It may be beneficial for clinicians to view maternal and/or fetal physiological signals during labor and/or delivery. Sometimes, the maternal and/or fetal physiological signals (such as biopotential or ECG signals) collected via electrodes that monitor maternal and/or fetal cardiac activity may be “lost” at some point during labor and the system may instead sense the maternal and/or fetal cardiac signals using acoustic sensors. For example, acoustic sensors may be able to sense heart sounds that are indicative of the cardiac activity. This means that the system described herein may provide a more seamless real-time monitoring of maternal and/or fetal cardiac activity as compared with systems that do not integrate signals sensed via electrodes and signals sensed via acoustic sensors to monitor cardiac activity.

In some examples, the acoustic sensors and the electrodes may be located in an array such that the sensors are located on an abdomen (e.g., torso) of the patient. In some examples, the sensor array may include the acoustic sensors located between electrodes. The electrodes and the acoustic sensors may be spread out across the abdomen in order to provide broad sensing coverage of the area where the fetus is located. Spreading out the sensors over a wide area increases an ability to sense cardiac activity of the fetus as compared with systems that do not spread sensors over a wide area, because the location and orientation of the fetus within the uterus may be different depending on the patient and the location and orientation of the fetus within the uterus may change during the course of the pregnancy. The wearable device that includes the sensor array may, in some examples, include a wearable textile garment that places the sensors on the abdomen. In some examples, the wearable device that includes the sensor array attaches to the abdomen using adhesive or other techniques for attaching the array to the patient without using a wearable garment.

The system may include one or more other sensors in addition to or alternatively to the electrodes and the acoustic sensors. For example, the other sensors may include one or more temperature sensors and one or more optical sensors. The one or more temperature sensors may measure a body temperature of the patient and/or a temperature of the environment around the patient. The one or more optical sensors may measure one or more parameters by emitting light waves and receiving reflected light waves based on emitting the light waves. For example, the one or more optical sensors may generate signals indicating a color of the fetus. The one or more optical sensors may additionally or alternatively generate tissue oxygen saturation (StO₂) signals, pulse oximetry (SpO₂) signals, photoplethysmography (PPG) signals, or any combination thereof. Based on one or more signals generated by the optical sensors, the system may be configured to determine a blood pressure of the mother and/or the fetus.

Based on one or more signals generated by the sensors of the wearable device, the system may be configured to identify and monitor one or more contractions of the uterus. It may be important to identify and monitor contractions during pregnancy and labor. There are several different kinds of contractions including true labor uterine contractions that occur during labor and result in delivery of the fetus, and Braxton-Hicks contractions that prepare the uterine muscle for delivery. Braxton-Hicks contractions can occur in any direction, occur sporadically and do not increase in strength. They are irregular in duration, frequency, and intensity, are unpredictable and non-rhythmic and are more uncomfortable than painful. True labor consists of contractions at regular intervals. True labor uterine contractions are directional, starting at the fulcrum of the uterus and moving downwards. As labor progresses, these contractions become stronger, and the time between each contraction decreases. One or more techniques described herein include analyzing signals generated by sensors on the sensor array in order to determine the strength of one or more contractions and/or the direction of one or more contractions. This information may be important for determining whether a contraction is a true labor uterine contraction, a Braxton-Hicks contraction, or another kind of contraction.

In some examples, the system may be configured to analyze one or more signals generated by sensors in order to provide real-time feedback during labor and/or delivery. For example, one or more sensor signals may indicate muscles that the patient is using during labor. This may allow clinicians to advise the patient based on whether the patient is using the proper muscles to deliver the baby.

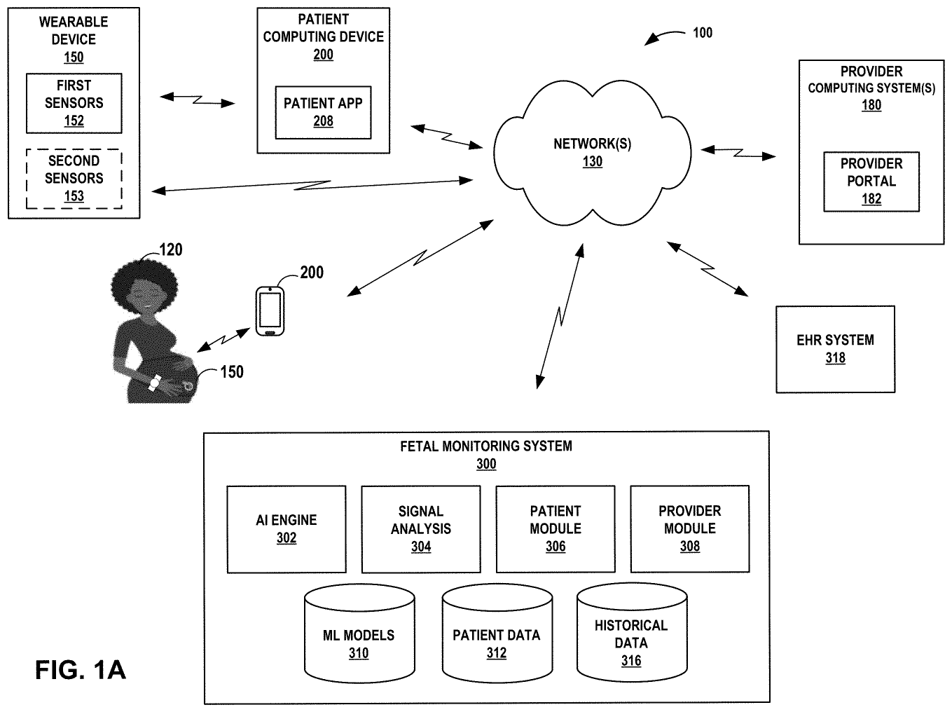

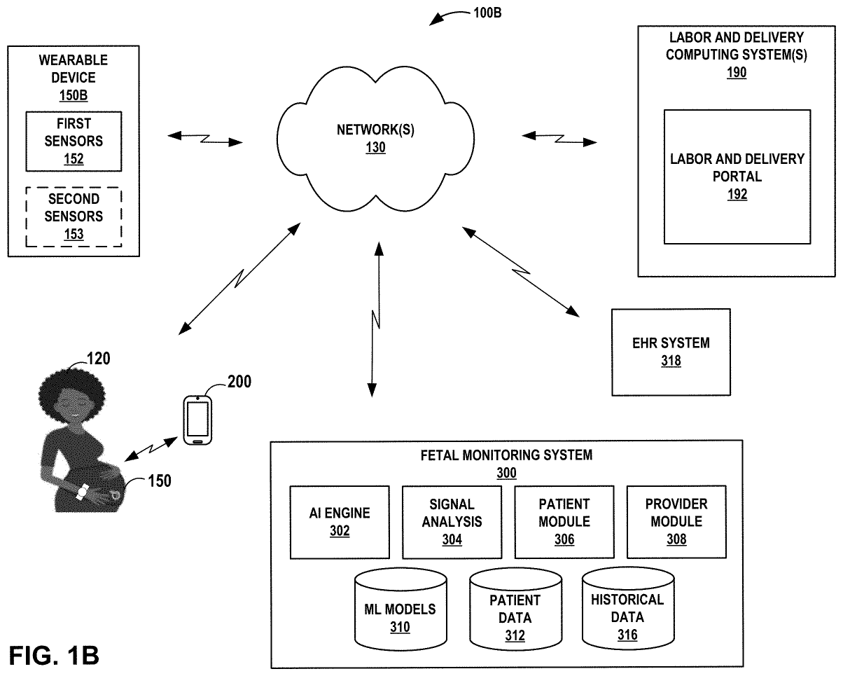

FIG. 1A is a diagram of an example system 100 for the acquisition and communication of patient data and prediction of maternal and/or fetal outcomes using trained machine learning model(s) in accordance with one or more techniques of the disclosure. In this example, system 100 includes a wearable device 150 including a first one or more sensors 152 and a second one or more sensors 153 (collectively, “sensors 152, 153”) configured to sense physiological signals of a patient 120 (a pregnant human mother and/or her fetus). Physiological signals measured by the first one or more sensors 152 may include, for example, maternal and/or fetal biopotential signals, such as ECG signals or other signals indicative of maternal and/or fetal cardiac activity, EMG signals indicative of uterine activity or contractions, etc. Physiological signals measured by the second one or more sensors 153 may include, for example, acoustic signals indicating one or more parameters of the patient and/or the fetus, optical signals indicating one or more parameters of the patient and/or the fetus, temperature signals, or any combination thereof. System 100 further includes at least one patient computing device 200, provider computing system 180, and a cloud-based fetal monitoring system (FMS) 300.

In some examples, system 100 of FIG. 1A is used to monitor one or more maternal and/or fetal physiological signals during pregnancy. For example, a pregnant mother may use wearable device 150 at home or other non-clinical environment for the acquisition of maternal and/or fetal physiological signals. These signals are then transmitted to patient computing device 200 (such as, for example, a smart phone running a patient application or patient “app”), which in combination with a cloud-based remote monitoring system (e.g., telehealth and/or telemedicine system, such as FMS 300 and/or provider computing system 180), may improve the mother's comfort and peace of mind during pregnancy. The techniques may be used to monitor the health of prenatal and postpartum patients in a remote monitoring setting.

The techniques of the disclosure may also be used during labor and delivery in addition to or instead of a traditional cardiotocography (CTG) monitoring device in clinical/hospital environment. An example labor and delivery computing system 190 is shown and described with respect to FIG. 1B.

In some examples, wearable device 150 includes a wearable (e.g., a garment or a band) configured to be worn on or about the torso of patient 120, the first one or more sensors 152 and the second one or more sensors 153 affixed or embedded in the wearable, a communications interface, and a controller. The first one or more sensors 152 are configured to sense one or more biometric signals, such as one or more biopotential signals of the mother and/or the fetus, such as ECG and/or EMG signals. In some examples, each sensor of the first one or more sensors 152 is configured to collect a respective electrical biopotential signal of a set of electrical biopotential signals. In some examples, an electrical biopotential signal corresponding to a sensor of the first one or more sensors 152 represents indicates an electrical potential of a tissue area of patient 120 proximate to the respective sensor. Electrical potential in a tissue area may, in some examples, indicate muscle activity in the tissue area.

In some examples, the first one or more sensors 152 are arranged on the wearable band of wearable device 150 such that when the wearable band is worn about the torso of the patient 120, each sensor of the set of sensors is located proximate to a respective location on the torso of the patient. In some examples, the one or more sensors of the first one or more sensors 152 are spread across an anterior of an abdomen of patient 120 such that the sensors form an array. For example, there may be one or more sensors of the first one or more sensors 152 located proximate to an upper abdomen of patient 120 and there may be one or more sensors of the first one or more sensors 152 located proximate to a lower abdomen of patient 120. In some examples, there may be one or more sensors of the first one or more sensors 152 located on a left portion of the abdomen of patient 120 and there may be one or more sensors of the first one or more sensors 152 located on a right portion of the abdomen of patient 120. One or more sensors of the first one or more sensors 152 may be located on a posterior of the abdomen of patient 120 and/or a side of the abdomen of patient 120. In any case, the first one or more sensors 152 may be spread across the abdomen of the patient 120 so that electrical potential signals collected by one or more sensors 152 indicate a potential of a range of different locations within the abdomen of patient 120.

The second one or more sensors 153 are configured to sense one or more signals that indicate other biomedical parameters of the patient and/or the mother. In some examples, the first one or more sensors 152 include electrodes that are configured to sense signals indicating a value of an electrical potential between two or more electrodes, a value of an electrical potential at a single electrode, an impedance at one or more electrodes, or any combination thereof. In some examples, the second one or more sensors 153 include one or more acoustic sensors, one or more optical sensors, one or more temperature sensors, or any combination thereof. The second one or more sensors 153 may generate signals indicative of cardiac activity of the patient and/or the fetus, respiratory activity of the patient and/or the fetus, muscle activity of the patient and/or the fetus, a temperature of the patient, a color of the fetus, or any combination thereof. In some examples, the physiological data sensed by the first one or more sensors 152 includes maternal and/or fetal ECG or heart rate data; however, the disclosure is not limited in this respect. Wearable device 150 is configured to wirelessly communicate sensor data generated by sensors 152, 153 for receipt by at least one computing device, such as patient computing device 200. The wearable device controller is configured to control signal acquisition from the one or more sensors and to control wireless communication of the sensor data.

Patient computing device 200 is configured for wireless communication with wearable device 150. For example, patient computing device 200 wirelessly receives the sensor data transmitted by wearable device 150. In some examples, patient computing device 200 may include one or more personal computing devices of the patient. For example, patient computing device 200 may include a mobile computing device (e.g., smartphone, tablet, or laptop computer), a desktop computer, a smartwatch, etc. Computing device 200 and wearable device 150 may communicate using, for example, the Bluetooth® or Bluetooth® Low Energy (BLE) protocols, near field communication (NFC), Wi-Fi protocols, Zigbee®, or any other form of wireless and/or wired communication.

In some examples, patient computing device 200 includes a patient application 208 stored in a memory or other data storage device of patient computing device 200 as a computer-readable medium comprising instructions that when executed by patient computing device 200, generates one or more interactive pages for display on a user interface of patient computing device 200. The one or more interactive pages guide the patient through a monitoring session during which physiological signals are acquired by wearable device 150 and corresponding sensed patient data is communicated from wearable device 150 to patient computing device 200.

Patient computing device 200 is further configured to communicate with a variety of other devices or systems via network(s) 130. For example, computing device 200 may be configured to communicate with one or more computing systems, e.g., provider computing system 180 and/or FMS 300.

FMS 300 includes an AI engine 302, a signal analysis module 304, a patient module 306, and a provider module 308. FMS 300 further includes or is associated with one or more databases or other storage device(s) that store one or more stored machine learning (ML) model(s) 310, patient data 312, sensor data 314, and historical patient data 316. Sensor data 314 includes the raw data representative of the biopotential signals detected by wearable device 150 during one or more patient monitoring sessions. Patient data 312 includes, for each of a plurality of patients, identification information corresponding to the patient, processed sensor data analyzed or generated by FMS 300 corresponding to one or more patient monitoring sessions, and/or one or more predicted outcomes corresponding to the one or more patient monitoring sessions. Historical patient data 316 includes historical maternal and/or fetal patient data associated with a plurality of patients. FMS 300 executes provider module 308 to provide remote provider-facing fetal monitoring services that support healthcare provider interaction with FMS 300 via provider portal 182 of provider computing system(s) 180. Similarly, FMS 300 executes patient module 306 to provide remote patient-facing fetal monitoring services that support patient interaction with FMS 300 via patient application 208 of patient computing device 200.

In accordance with one or more techniques of the disclosure, AI engine 302 of FMS 300 is configured to train one or more machine learning (ML) model(s) 310 based on historical patient data 316 associated with a plurality of patients. In some examples, ML model(s) 310 may generate information corresponding to maternal health, fetal health, and/or pregnancy status. AI engine 302 may, in some examples, determine, based analyzing patient data for patient 120 using one or more ML models 310 trained with the historical patient data 316, one or more maternal and/or fetal outcome predictions for the pregnant patient. Example systems and methods of training of the one or more machine learning models and or prediction of one or more maternal and/or fetal outcomes are described in U.S. patent application Ser. No. 17/457,206, filed Dec. 1, 2021 and U.S. patent application Ser. No. 18/145,562, filed Dec. 22, 2022, each of which is incorporated by reference herein in its entirety.

Patient computing device(s) 200 may transmit data, including patient data received from wearable device 150, to computing system(s) 180 and/or FMS 300 via network(s) 130. The data may include sensed patient data, e.g., values of one or more biopotential signals, such as ECG and/or EMG signals, sensed by wearable device 150 and other physiological signals or data sensed or otherwise determined by wearable device 150 and/or patient computing device(s) 200. FMS 300 may retrieve data regarding patient(s) from one or more sources of electronic health records (EHR) system 318 (which may also be referred to as electronic medical records, EMR) via network 130. EHR system 318 may include data regarding historical (e.g., baseline) patient data, previous health events and treatments, preexisting conditions, disease states, comorbidities, demographics, height, weight, and body mass index (BMI), as examples, of patients. FMS 300 may use data from EHR system 318 to configure algorithms implemented by wearable device 150, patient computing device 200 and/or FMS 300 to control acquisition of the sensed biopotential signals from wearable device 150 during a monitoring session and/or to predict maternal and/or fetal outcomes based on patient data acquired during a monitoring session for a patient.

Network(s) 130 may include, for example one or more local area networks (LANs), wireless local area networks (WLANs), virtual private networks (VPNs), wide area networks (WANs), the Internet, etc. Network(s) 130 may include one or more computing devices, such as one or more non-edge switches, routers, hubs, gateways, security devices such as firewalls, intrusion detection, and/or intrusion prevention devices, servers, cellular base stations and nodes, wireless access points, bridges, cable modems, application accelerators, or other network devices. Network(s) 130 may include one or more networks administered by service providers and may thus form part of a large-scale public network infrastructure, e.g., the Internet. Network(s) 130 may provide computing devices and systems, such as those illustrated in FIG. 1A, access to the Internet, and may provide a communication framework that allows the computing devices and systems to communicate with one another. In some examples, network(s) 130 may include a private network that provides a communication framework that allows the computing devices and systems illustrated in FIG. 1A to communicate with each other but isolates some of the data flows from devices external to the private network for security purposes. In some examples, the communications between the computing devices and systems illustrated in FIG. 1A are encrypted.

Provider computing system 180 includes one or more computing devices used by providers (e.g., physicians, physician assistants, nurses, nurse midwives, pharmacists, therapists, clinical support staff, etc.) to view patient data gathered or generated during one or more patient monitoring sessions, including one or more maternal and/or fetal outcome predictions associated with the patient monitoring sessions, for one or more patients. For example, provider computing system 180 may include a provider portal 182 stored in a memory or other data storage device of provider computing system 180 as a computer-readable medium comprising instructions that when executed by provider computing system 180 generates one or more interactive pages for display on a user interface of provider computing system 180 that allow health care providers to view raw and/or processed patient data or other data generated by analysis of the patient data, including one or more predicted maternal and/or fetal outcomes, for one or more patients.

In some examples, FMS 300 is configured to receive, from sensors 152, biometric data indicative of a muscle contraction of a patient over a period of time. In some examples, the biometric data may include one or more biopotential signals (e.g., ECG signals). FMS 300 may, in some examples, identify determine, based on the set of biopotential signals, a set of electrical potential vector signals, wherein each electrical potential vector signal of the set of electrical potential vector signals represents a difference between an electrical signal of the set of electrical signals collected by a reference sensor of sensors 152 and an electrical signal of the set of electrical signals collected by another sensor of sensors 152. That is, each electrical potential vector signal of the set of electrical potential vector signals may represent a difference between an electrical potential of a tissue area proximate to the reference sensor of sensors 152 and an electrical potential of a tissue area proximate to another sensor of sensors 152.

FMS 300 is configured to determine, based on the biometric data, a muscle contraction vector indicating a direction of a muscle contraction over the period of time. A machine learning model of machine learning models 310 may, in some cases, determine the muscle contraction vector. In some examples, the direction of the muscle contraction vector represents a direction relative to the body of the patient. For example, the distal direction may represent a movement towards the feet of the patient 120 as the patient is standing straight up or lying down with legs and torso straight. The proximal direction may represent a movement towards the feet of the patient 120 as the patient is standing straight up or lying down with legs and torso straight. Diagonal directions may represent any direction that forms an angle with a longitudinal axis of the patient 120 when the patient is lying down with legs and torso straight.

The muscle contraction vector may indicate the direction of a muscle contraction over a period of time corresponding to the biometric data received by FMS 300. For example, FMS 300 may receive biometric data that is collected over a period of time. FMS 300 may process the biometric data to determine the muscle contraction vector. The muscle contraction vector may, in some examples, indicate that a muscle contraction or a series of muscle contractions of patient 120 are moving substantially in a distal direction. In some examples, muscle contraction vector may indicate that a muscle contraction or a series of muscle contractions of patient 120 are moving in a direction or directions that is different than the distal direction. In

FMS 300 may, in some cases determine, based on the biometric data, a likelihood that the muscle contraction comprises a true labor uterine contraction. In some examples, a machine learning model of machine learning models 310 may determine the likelihood that the muscle contraction comprises a true labor uterine contraction. FMS 300 may train the machine learning model of machine learning models 310 training data that includes one or more sets of biometric data known to be associated with true labor uterine contractions and one or more sets of biometric data known not to be associated with true labor uterine contractions. In some examples, the one or more sets of biometric data known not to be associated with true labor uterine contractions includes one or more sets of biometric data associated with uterine contractions other than true labor uterine contractions, such as Braxton-Hicks contractions.

In some examples, true labor uterine contractions include one or more characteristics that distinguish true labor uterine contractions from other kinds of uterine contractions. For example, true labor uterine contractions may consistently travel in the distal direction relative to the body of patient 120. Other uterine contractions that are not uterine contractions, such as Braxton-Hicks contractions, may travel in directions that differ substantially from the distal direction (e.g., the proximal direction, or in “sideways” directions that form significant angles with the distal direction). True labor uterine contractions may, in some examples, occur at regular intervals and occur closer together over time. That is, each pair of consecutive true labor uterine contractions of a sequence of true labor uterine contractions may be separated by approximately the same amount of time as one or more other pairs of consecutive true labor uterine contractions. In some examples, true labor uterine contractions may be stronger than one or more other kinds of uterine contractions. In some examples, a duration of true labor uterine contractions is within a range, such as between 30 seconds and 90 seconds. In some examples, one or more other kinds of uterine contractions have a duration that is shorter than 30 seconds.

FMS 300 may output, for display by a user device (e.g., provider portal 182), information corresponding to one or more muscle contractions of patient 120. For example, FMS 300 may output the muscle contraction vector indicating the direction of the muscle contraction over a period of time. Additionally, or alternatively, FMS 300 may output and the likelihood that the muscle contraction comprises a true labor uterine contraction. In some examples, FMS 300 may output a timeseries indicating a strength of one or more muscle contractions over a period of time. The user device may display the information corresponding to the one or more muscle contractions of patient 120.

In some examples, wearable device 150 may output the biometric data collected by first sensors 152 to FMS 300 via patient computing device 200, but this is not required. In some examples, wearable device 150 may output the biometric data collected by first sensors 152 to FMS 300 via network(s) 300 without outputting the biometric data to patient computing device 200. In some examples, FMS 300 may output information to patient computing device 200 based on analyzing the biometric data. In some examples, FMS 300 may output information to patient provider computing system(s) 180 based on analyzing the biometric data.

FIG. 1B is a diagram of an example system 100B for the acquisition and communication of patient data during labor and delivery in accordance with one or more techniques of the disclosure. In this example, system 100B includes a non-invasive wearable device 150B including a first one or more sensors 152 and a second one or more sensors 153 (collectively, “sensors 152, 153”) configured to sense physiological signals of a patient (a pregnant human mother and/or her fetus). Similar to that described above with respect to FIG. 1A, physiological signals generated by the first one or more sensors 152 may include, for example, maternal and/or fetal biopotential signals, such as ECG signals or other signals indicative of maternal and/or fetal cardiac activity, EMG signals indicative of uterine activity or contractions, etc. Physiological signals generated by the second one or more sensors 153 may include, for example, acoustic signals indicating one or more parameters of the patient and/or the fetus, optical signals indicating one or more parameters of the patient and/or the fetus, temperature signals, or any combination thereof.

System 100B further includes a labor and delivery (LD) computing system 190. LD computing system 190 is further configured to communicate with EHR system 318 and, in some examples, with a cloud-based fetal monitoring system (FMS) 300. In this example, system 100B of FIG. 1B is used for real time monitoring of one or more maternal and/or fetal physiological signals during labor and delivery. The techniques of the disclosure may be used in addition to or instead of a traditional cardiotocography (CTG) monitoring device in a clinical/hospital environment. Wearable device 150B is configured for communication with LD computing system 190. The communication may be wired or wireless. Examples of wireless communication that may be used include, but are not limited to, Bluetooth®, Bluetooth® Low Energy (BLE), Wi-Fi, radio frequency (RF) communication, infrared communication, Zigbee®, near-field communication (NFC), cellular or other types of mobile communication, satellite communication, and any other type of wireless communication. The communication may be direct or via one or more networks 130.

In some examples, wearable device 150B of FIG. 1B may include one or more of the same sensors 152, 153 as wearable device 150 of FIG. 1A. In some examples, wearable device 150B of FIG. 1B may further include one or more different sensors 152, 153 as wearable device 150 of FIG. 1A. In some examples, one or more of sensors 152, 153 of FIG. 1A may include sensors selected for monitoring during pregnancy at home or in a non-clinical setting, and one or more sensors 152, 153 of FIG. 1B may include sensors selected for real time monitoring during labor and delivery and/or in a hospital or other clinical setting.

In some examples, wearable device 150B of FIG. 1B includes a wearable element configured to be worn on or about the torso of a pregnant mother, the first one or more sensors 152 and the second one or more sensors 153 affixed or embedded in the wearable, a communications interface, and a controller. The first one or more sensors 152 are configured to sense one or more biopotential signals of the mother and/or the fetus, such as ECG and/or EMG signals. The second one or more sensors 153 are configured to sense one or more signals that indicate other biomedical parameters of the patient and/or the mother. In some examples, the first one or more sensors 152 include electrodes that are configured to sense signals indicating a value of an electrical potential between two or more electrodes, a value of an electrical potential at a single electrode, an impedance at one or more electrodes, or any combination thereof. In some examples, the second one or more sensors 153 include one or more acoustic sensors, one or more optical sensors, one or more temperature sensors, or any combination thereof. The second one or more sensors 153 may generate signals indicative of cardiac activity of the patient and/or the fetus, respiratory activity of the patient and/or the fetus, muscle activity of the patient and/or the fetus, a temperature of the patient, a color of the fetus, or any combination thereof. In some examples, the physiological data sensed by the first one or more sensors 152 includes maternal and/or fetal ECG or heart rate data. The physiological data sensed by the first one or more sensors 152 may further include EMG signals indicative of uterine contraction data. However, the disclosure is not limited in this respect. As described above, in some examples, wearable device 150B is configured to wirelessly communicate sensor data generated by sensors 152, 153 for receipt by at least one computing device, such as LD computing system 190. The wearable device controller is configured to control signal acquisition from the one or more sensors and to control wireless communication of the sensor data.

LD computing system 190 wirelessly receives the sensor data transmitted by wearable device 150B. LD computing system 190 may be further configured to communicate with a variety of other devices or systems via network(s) 130. For example, LD computing system 190 may be configured to communicate with one or more computing systems, e.g., EHR system 318 and/or FMS 300.

LD computing system 190 includes one or more computing devices used by providers (e.g., physicians, physician assistants, nurses, nurse midwives, pharmacists, therapists, clinical support staff, etc.) to view patient data gathered or generated for a patient during labor and delivery. For example, LD computing system 190 may include a LD portal 192 stored in a memory or other data storage device of LD computing system 190 as a computer-readable medium comprising instructions that when executed by LD computing system 190 allows health care providers to view data sensed in real time by wearable device 150 during labor and delivery. For example, LD system may display sensed signals and/or data including any one or more of maternal heart rate, fetal heart rate, maternal, uterine activity signals, uterine contraction data (e.g., start, end, duration, strength, resting time between contractions, contraction location and direction), and any other information sensed by wearable device 150 or data derived from the information sensed by wearable device 150.

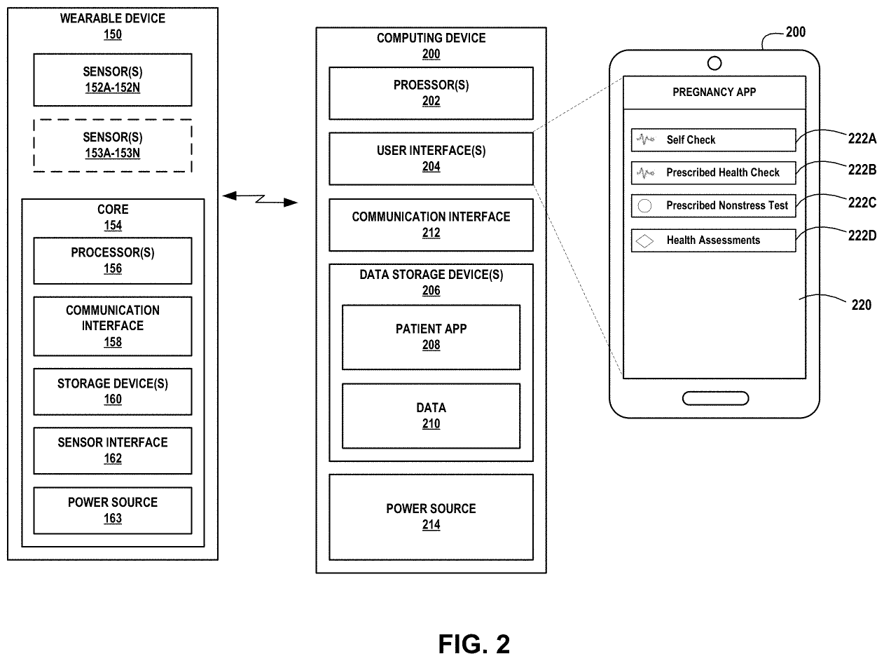

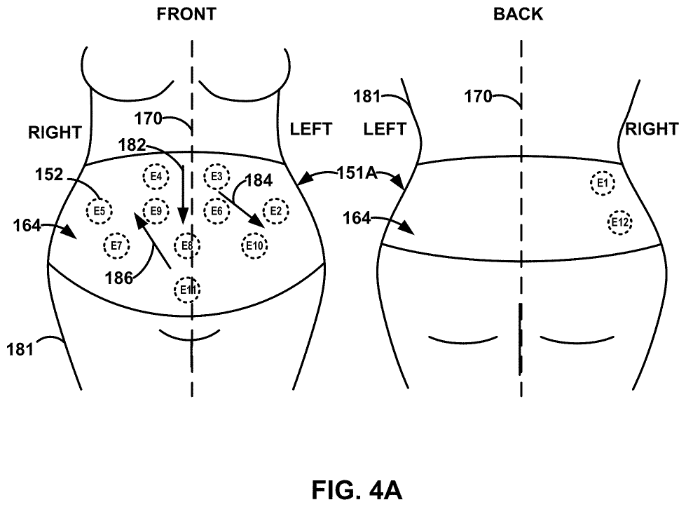

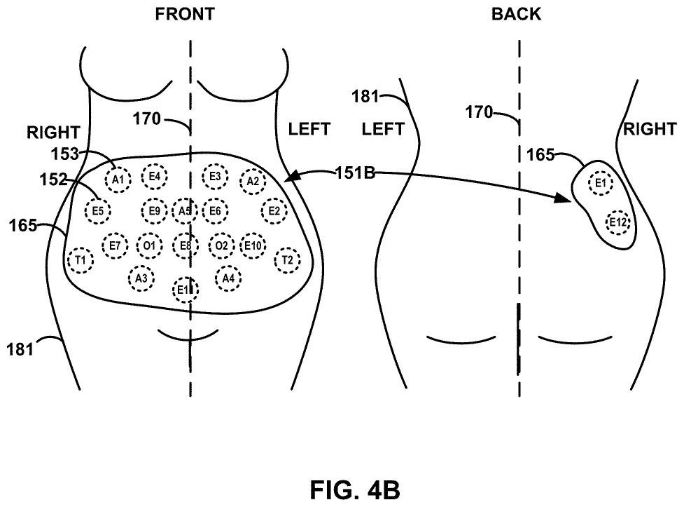

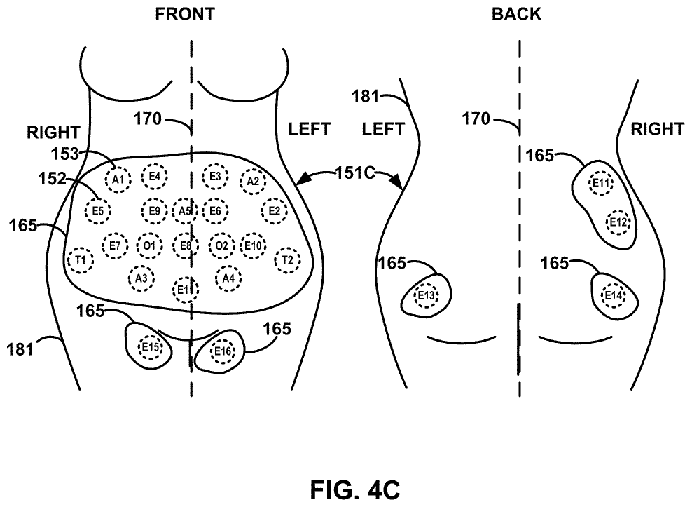

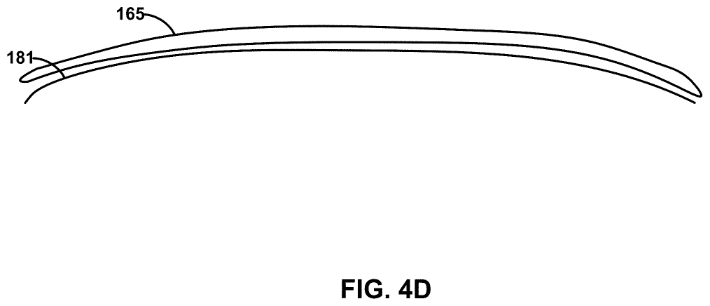

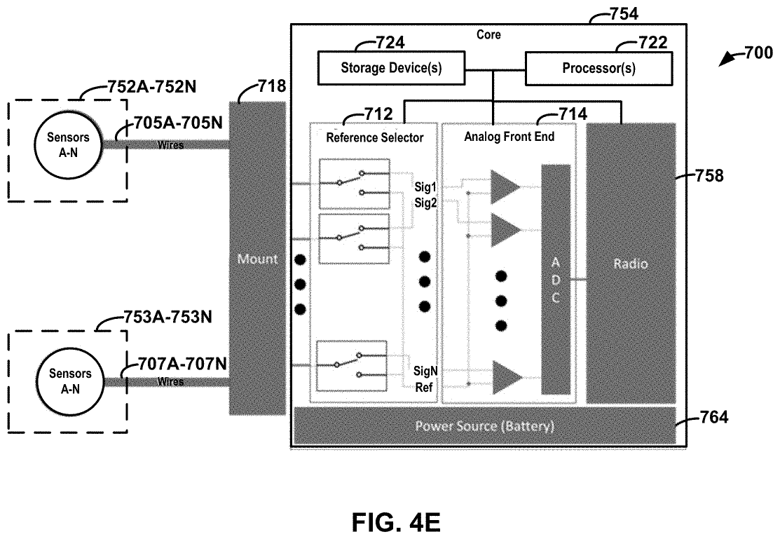

FIG. 2 is a more detailed block diagram of the electronic components of an example wearable device 150 and an example patient computing device 200 in accordance with one or more techniques of the disclosure. Wearable device 150 of FIG. 2 may be used to implement wearable device 150 of FIG. 1A and/or wearable device 150B or FIG. 1B. Wearable device 150 includes the first one or more sensors 152 configured to sense physiological signals of a patient, such as maternal and/or fetal biopotential signals, such as ECG and/or EMG signals. Wearable device 150 includes the second one or more sensors 153 configured to generate physiological signals of the patient, such as optical signals, audio signals, and temperature signals indicating one or more parameters of the mother and/or the fetus. In some examples, wearable device 150 includes a wearable (e.g., a garment or band 164 such as shown in FIG. 4A) including a plurality of electrodes or other sensing devices 152A-152N affixed to or embedded therein. In some examples, the wearable is a wearable element that is attached to the abdomen without comprising a garment.

In some examples, sensors 152, 153 are configured to sense maternal and/or fetal ECG signals. In other examples, one or more of the sensors 152, 153 may be configured to sense any one or more of a cardiotocography (CTG) signals, EMG signals, EMG myometrium signals, pulse oximeter signals, respiratory inductance plethysmography (RIP) (thoracic and abdominal) signals, acoustic signals, actigraphy signals, temperature information, accelerometer or movement information, photoplethysmography (PPG) (e.g., optical measurement for pulse rate and SpO₂), and/or any other biopotential or physiological signal or parameter of the patient.

Wearable device further includes control electronics that process the sensed physiological signals of the patient acquired by the first one or more sensors 152 and the second one or more sensors 153, and communicate the sensed patient data for receipt by patient computing device 200. In some examples, the control electronics are packaged in a core 154 configured to be removably connected to the wearable garment or band. To that end, core 154 includes one or more processors 156, a communication interface 158, storage devices 160, a sensor interface 162, and a power source 163 (e.g., one or more batteries). Sensor interface 162 includes circuitry configured to receive sensor data corresponding to the sensed physiological signals from the first one or more sensors 152 and the second one or more sensors 153. Communication interface 158 is configured to support wireless communication between wearable device 150 and one or more computing devices, such as patient computing device 200. Storage devices 160 include one or more hardware memories or other data storage devices configured to store executable control instruction and/or raw sensor data associated with one or more monitoring sessions. Wearable device 150 may store sensor data temporarily during each monitoring session for wireless transmission to a computing device, or wearable device may store sensor data associated with multiple monitoring sessions for later transmission to a computing device.

Patient computing device 200 includes one or more processor(s) 202, a user interface 204, communication interface 212, data storage devices 206, and a power source 214 (e.g., one or more batteries). In some examples, patient computing device 200 may include one or more personal computing devices of the patient. For example, patient computing device 200 may include a mobile computing device (e.g., smartphone, tablet, or laptop computer), a desktop computer, a smartwatch, etc. Communication interface 212 of patient computing device 200 is configured for wireless communication with wearable device 150. For example, communication interface 212 and communication interface 158 of wearable device 150 may be configured to communicate using, for example, the Bluetooth® or Bluetooth® Low Energy (BLE) protocols, near field communication (NFC), or any other form of wireless communication.

Patient computing device 200 includes a patient application 208 stored in data storage device(s) 206. For example, patient application 208 may include a computer-readable medium comprising instructions that when executed by one or more processor(s) 202 of patient computing device 200 generates one or more interactive pages for display on a user interface 204 of patient computing device 200 that guide the patient through a monitoring session during which physiological signals are acquired by wearable device 150 and corresponding sensor data is communicated from wearable device 150 to patient computing device 200. As shown in the example of FIG. 2, example patient computing device 200 includes a touch screen display 220 on which one or more interactive pages of a guided patient monitoring session are displayed. Each interactive page may include one or more user interface elements, such as user interface elements 222A-222D, by which a user may interact with patient application 208 (and thus with wearable device 150 and/or FMS 300) to conduct one or more monitoring sessions including a self check, prescribed health check, prescribed nonstress test, and/or one or more health assessments, such as one or more mental health assessments, social determinants of health assessments, socio-economic assessments, etc.

Communication interface 212 of patient computing device 200 is further configured to communicate with a variety of other devices or systems via network(s) 130 (see FIG. 1A). For example, computing device 200 may be configured to communicate with one or more computing systems, e.g., one or more of provider computing system 180 and/or FMS 300.

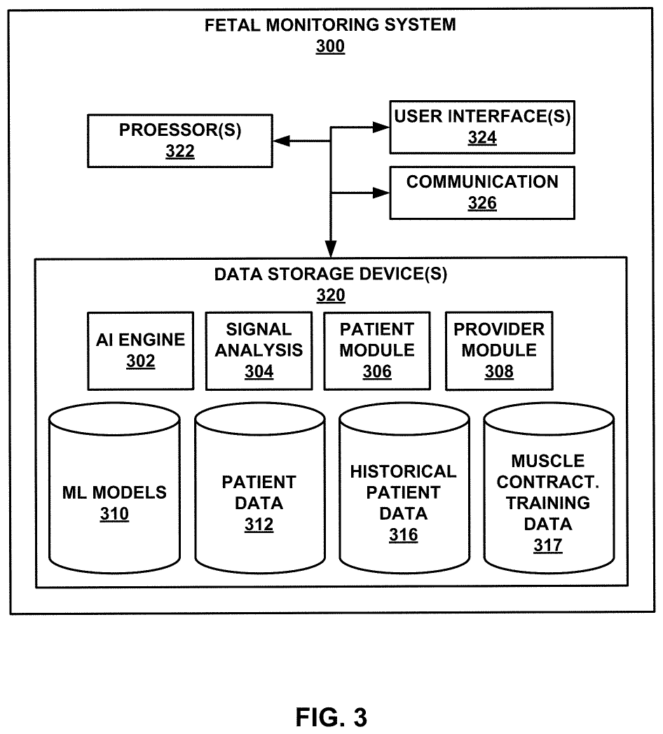

FIG. 3 is a block diagram of an example fetal monitoring system (FMS) 300 in accordance with one or more techniques of the disclosure. FMS 300 includes one or more processors 322, user interfaces 324 by which one or more users may interact with FMS 300, communication interfaces 326 which provide for communication with one or more computing devices such as patient computing device 200 and/or provider computing systems 180, and one or more data storage devices 320. Data storage devices 320 include storage for one or more computing modules including AI engine 302, signal analysis module 304, patient module 306, and provider portal module 308. FMS 300 further includes or is associated with one or more databases or other storage device(s) that store one or more stored machine learning (ML) model(s) 310, patient data 312, and historical patient data 316. Patient data 312 includes, for each of a plurality of patients, biopotential or other physiological patient data sensed by wearable device 150 during one or more patient monitoring sessions, biometric data associated with the patient, and/or patient data obtained during one or more health assessment sessions. Patient data 312 also includes, for each of a plurality of patients, identification information corresponding to the patient, processed sensor data analyzed or generated by FMS 300 corresponding to one or more patient monitoring sessions, and/or one or more predicted outcomes corresponding to the one or more patient monitoring sessions. Historical patient data 316 includes historical patient data associated with a plurality of patients. Processor(s) 322 of FMS 300 execute provider module 308 to provide remote provider-facing fetal monitoring services that support healthcare provider interaction with FMS 300 via provider portal 182 of provider computing system(s) 180. Similarly, processors 322 of FMS 300 execute patient module 306 to provide remote patient-facing fetal monitoring services that support patient interaction with FMS 300 via patient application 208 of patient computing device 200.

Signal analysis module 304 may apply one or more signal processing or preprocessing techniques to the raw sensor data. For example, signal analysis module 304 may apply normalization, denoising, filtering, artifact detection and/or artifact correction to any one or more of the sensed signal data received from the wearable device 150. Signal analysis modules may also perform feature extraction for the sensed biopotential signals including for example, extraction of a fetal ECG signal from a mixed maternal-fetal ECG signal, identification of one or more features of the maternal and/or fetal ECG signals including, for example, one or more features of the P wave, QRS complex, T wave, PQ interval, QRS duration, QT interval, RR interval, or other feature indicative of the electrical activity of the heart (e.g., start, end, duration, amplitude, peak-to-peak information, morphology, etc.). Signal analysis module 304 may further extract one or more features of the maternal and/or fetal heart rate signals including, for example, baseline heart rate, baseline variability, fetal heart rate variability, number of accelerations per second, number of early, late, and variable decelerations per second, number of prolonged decelerations per second, sinusoidal pattern, etc.

FMS 300 executes provider module 308 to provide remote provider-facing fetal monitoring services that support healthcare provider interaction with FMS 300 via provider portal 182 of provider computing system(s) 180. Similarly, FMS 300 executes patient module 306 to provide remote patient-facing fetal monitoring services that support patient interaction with FMS 300 via patient application 208 of patient computing device 200.

In accordance with one or more techniques of the disclosure, AI engine 302, when executed by processors 322 of FMS 300, is configured to train one or more machine learning (ML) models of ML models 310 based on historical patient data 316 associated with a plurality of patients to generate one or more maternal and/or fetal outcome predictions. AI engine 302, when executed by processors 322, is further configured to determine, based on processing patient data for a pregnant mother using one or more ML models 310 trained with the historical patient data corresponding to a plurality of patients 316, one or more maternal and/or fetal outcome predictions for the pregnant mother.

In accordance with one or more techniques of the disclosure, AI engine 302, when executed by processors 322 of FMS 300, is configured to train one or more machine learning (ML) models of ML models 310 based on historical patient data 316 and/or muscle contraction training data 317 associated with a plurality of patients to generate information relating to uterine muscle contractions of a patient (e.g., patient 120). In some examples, muscle contraction training data 317 may include biopotential signals associated with uterine muscle contractions of one or more patients. In some examples, muscle contraction training data 317 may include a first one or more datasets known to be associated with true labor uterine contractions. In some examples, muscle contraction training data 317 may include a second one or more datasets known to be associated with uterine contractions other than true labor uterine contractions, such as Braxton-Hicks contractions.

Although in the examples described herein FMS 300 is described as performing the training of the ML models 310 and/or application of the models 310 to predict one or more maternal or fetal outcomes and/or output information corresponding to uterine contractions of a patient, it shall be understood that some or all of the functions described herein as being performed by FMS 300 may be performed by any one or more of wearable device 150, patient computing device 200, provider computing system 180, or any other remote, local or distributed computing device or system, and that the disclosure is not limited in this respect.

FMS 300 may, in some examples, receive from sensors 152, biometric data indicative of a muscle contraction of patient 120 over a period of time. For example, FMS 300 may receive a set of electrical signals from sensors 152, wherein each electrical signal of the set of electrical signals corresponds to a respective sensor of sensors 152. The set of electrical signals may, in some cases, form a set of electrical potential vector signals. Each electrical potential signal of the set of electrical potential signals may represent a difference between an electrical signal corresponding to a reference sensor of sensors 152, and an electrical signal corresponding to another sensor of sensors 152. In some examples, FMS 300 may determine the set of electrical potential signals based on the set of electrical signals received from sensors 152 of wearable device 150. In some examples, wearable device 150 may determine the set of electrical potential signals based on the set of electrical signals collected by sensors 152 and output the set of electrical potential signals to FMS 300.

Processors 322 may execute a machine learning model of ML models 310 to generate, based on biometric data received from wearable device 150, information corresponding to one or more muscle contractions of patient 120. For example, the machine learning model may analyze a set of electrical potential vector signals to determine a muscle contraction vector indicating a direction of one or more muscle contractions over a period of time. Additionally, or alternatively, the machine learning model may analyze a set of electrical potential vector signals to determine a likelihood that a muscle contraction comprises a true labor uterine contraction. In some examples, the machine learning model may analyze a set of electrical potential vector signals to generate a time series indicating a strength of one or more muscle contractions over a period of time.

FMS 300 may output, for display by a user device, information corresponding to one or more muscle contractions. For example, FMS 300 may output a muscle contraction vector indicating a direction of one or more muscle contractions over a period of time, a likelihood that a muscle contraction comprises a true labor uterine contraction, a time series indicating a strength of one or more muscle contractions over a period of time, or any combination thereof. In some examples, FMS 300 may output information corresponding to a muscle contraction for display by user interface(s) 324. In some examples, FMS 300 may output information corresponding to a muscle contraction for display by a user interface of provider portal 182. In some examples, FMS 300 may output information corresponding to a muscle contraction for display by a user interface of labor and delivery portal 192. In some examples, FMS 300 may output information corresponding to a muscle contraction for display by touch screen display 220 of patient comp.

FIG. 4A is a diagram of a first example wearable device 151A including a first one or more sensors 152 (labeled E1-E12 and referred to generally as sensors 152 or electrodes 152) embedded or affixed to a wearable garment 164, in accordance with one or more techniques of the disclosure. First example wearable device 151A may be an example of wearable device 150 of FIGS. 1A, 1B, and 2. In this example, the sensor configuration includes a total of twelve electrodes E1-E12 affixed to or embedded within garment 164 such that, when properly worn about the torso of a patient 181, the electrodes 152 are positioned about the torso of patient 181. One or more of the electrodes 152 may be positioned on the front, either side or back of the patient 181. As shown in FIG. 4A, in some examples, the sensor configuration on garment 164 is such that two electrodes are positioned on the back of patient 181 (E1 and E12 in this example). The number and configuration of sensors 152 on the wearable garment 164 may vary from that shown in FIG. 4A, and the disclosure is not limited in this respect.

In order to capture maternal and fetal biopotential signals of sufficient quality, sensors 152 should provide good contact with the patient's skin, minimize sensor movement relative to the skin, and reduce signal noise from light movements of the patient. In some examples, sensors 152 include SilverBumps® dry electrodes available from Orbital Research, Inc. Example wearable garments that may be used to implement wearable device 151A are described in U.S. Pat. No. 9,579,055, issued Feb. 28, 2017, which is incorporated by reference herein in its entirety.

In other examples, instead of or in addition to dry electrodes, wearable device 151A may include any other type of sensing material or device to acquire the biopotential signals data, such as nanotechnology sensing devices, textile or silicon-based dry electrodes, nanotube sensors, cardiotocography (CTG) doppler transducers for acquiring signals associated with uterine contractions, and/or any other sensor that may be used to capture maternal and/or fetal biopotential signals.

In some examples, each electrode of electrodes 152 is configured to collect an electrical signal that indicates an electrical potential of a tissue area proximate to the respective electrode. For example, electrode E1 may collect an electrical signal that indicates an electrical potential of a tissue area proximate to electrode E1, electrode E2 may collect an electrical signal that indicates an electrical potential of a tissue area proximate to electrode E2, electrode E3 may collect an electrical signal that indicates an electrical potential of a tissue area proximate to electrode E3, electrode E4 may collect an electrical signal that indicates an electrical potential of a tissue area proximate to electrode E4, and so on.

In some examples, an electrical potential corresponding to a tissue area may indicate muscle activity in the tissue area. For example, an increase in electrical potential in a tissue area may indicate an increase in muscle activity (e.g., an increase in the existence and/or strength of muscle contraction) in the tissue area. In some examples, one or more electrical potential signals that indicate muscle activity comprise EMG signals.

In some examples, the electrical signals collected by electrodes 152 may form a set of electrical potential vector signals. An electrical potential vector signal may represent a difference between a magnitude of an electrical signal collected by a reference electrode of electrodes 152 and a magnitude of an electrical signal collected by another electrode of electrodes 152. In some examples, electrode E11 may represent a reference electrode, but this is not required. Any of electrodes E1-E12 may serve as a reference electrode. In some examples, an electrical potential vector signal may comprise a difference between a magnitude of an electrical signal collected by reference electrode E11 and a magnitude of an electrical signal collected by electrode E1, an electrical potential vector signal may comprise a difference between a magnitude of an electrical signal collected by reference electrode E11 and a magnitude of an electrical signal collected by electrode E2, an electrical potential vector signal may comprise a difference between a magnitude of an electrical signal collected by reference electrode E11 and a magnitude of an electrical signal collected by electrode E3, and so on.

Since electrodes E1-E12 are affixed to garment 164 such that electrodes E1-E12 are spread across an abdomen and lower back of patient 181 when patient 181 wears the garment 164, electrical potential vector signals corresponding to electrodes E1-E12 may indicate one or more differences in electrical potential corresponding to different areas of the patient's abdomen and lower back. For example, an electrical potential vector signal indicating a difference in the electrical potential corresponding to electrode E11 and the electrical potential corresponding to electrode E4 may be different from an electrical potential vector signal indicating a difference in the electrical potential corresponding to electrode E11 and the electrical potential corresponding to electrode E8, because electrode E4 is located higher on the patient's abdomen than electrode E4. This means that the electrical potential vector signal corresponding to electrode E4 may indicate muscle activity occurring higher on the abdomen of patient 181 as compared with the electrical potential vector signal corresponding to electrode E8.

Uterine contractions may propagate in one or more directions relative to the body of the patient. In some examples, a direction of a uterine contraction may constitute a movement of a uterine contraction across a portion of the patient relative to a longitudinal axis 170 of the patient's body. The longitudinal axis of the patient's body may extend through a center of the body from the head to the feet when the patient is standing or lying down with legs, neck, and torso straight. When a uterine contraction moves, muscle activity may occur at a first location on the patient's body, muscle activity may occur at a second location on the patient's body proximate to the first location after the muscle activity occurs at the first location, muscle activity may occur at a third location on the patient's body proximate to the second location after the muscle activity occurs at the second location, and so on. The direction of the muscle contraction may, in some examples, comprise an angle that a movement vector of the muscle contraction forms with longitudinal axis 170.

In some examples, a true labor uterine contraction may move in a first direction 182 that is parallel to or nearly parallel to longitudinal axis 170. First direction 182 is an example of a direction that is parallel or nearly parallel to longitudinal axis 170. For example, first direction 182 may represent a direction moving from a higher portion of the patient's abdomen to a lower portion of the patient's abdomen. A uterine contraction moving in the first direction 182 may, in some cases, start at an upper portion of the patient's abdomen and move, substantially parallel to longitudinal axis 170, towards a lower portion of the patient's abdomen.

In some examples, a true labor uterine contraction may move in a second direction 184 that is at an angle relative to longitudinal axis 170. As seen in FIG. 4A, second direction 184 is at an angle with longitudinal axis 170. Second direction 184 extends downwards and leftwards relative to the patient's body. That is, second direction 184 is diagonal relative to longitudinal axis 170 and does not extend parallel or substantially parallel to longitudinal axis 170. In some examples, a true labor uterine contraction may move in a third direction 186 that is at an angle relative to longitudinal axis 170. As seen in FIG. 4A, third direction 186 is at an angle with longitudinal axis 170. Third direction 186 extends upwards and rightwards relative to the patient's body. That is, third direction 186 is diagonal relative to longitudinal axis 170 and does not extend parallel or substantially parallel to longitudinal axis 170.

First direction 182, second direction 186, and third direction 184 are not the only directions in which uterine contractions can travel, and the techniques of this disclosure are not limited to first direction 182, second direction 186, and third direction 184. A uterine contraction may propagate in any direction relative to longitudinal axis 170. In some examples, a muscle contraction may change directions as it propagates. For example, a muscle contraction may begin traveling in first direction 182 and then pivot to traveling in second direction 184 or any other direction.

In some examples, a set of electrical potential vector signals corresponding to electrodes E1-E12 may indicate one or more directions of a muscle contraction. For example, when a muscle contraction travels in direction 182, an electrical potential vector signal corresponding to electrode E3 and an electrical potential vector signal corresponding to electrode E4 may first indicate muscle activity. After the electrical potential vector signal corresponding to electrode E3 and the electrical potential vector signal corresponding to electrode E4 indicates muscle activity, electrical potential vector signals corresponding to electrodes E2, E5, E6, and E9 may indicate muscle activity. After the electrical potential vector signal corresponding to electrodes E2, E5, E6, and E9 indicate muscle activity, electrical potential vector signals corresponding to electrodes E7, E8, and E10 may indicate muscle activity. That is, the set of electrical potential vector signals corresponding to electrodes E1-E12 may indicate that a muscle contraction is traveling in direction 182 when muscle activity begins at an upper portion of the patient's abdomen proximate to electrodes E3 and E4 and travels towards a lower portion of the patient's abdomen proximate to electrodes E7, E8, and E10.

In some examples, when a muscle contraction travels in direction 182, an electrical potential vector signal corresponding to electrode E3 and an electrical potential vector signal corresponding to electrode E6 may first indicate muscle activity. After the electrical potential vector signal corresponding to electrode E3 and the electrical potential vector signal corresponding to electrode E6 indicates muscle activity, electrical potential vector signals corresponding to electrodes E2 and E10 may indicate muscle activity. This may indicate that the muscle contraction is traveling in the downwards and leftwards direction of second direction 184. Since electrodes E1-E12 are spread across an abdomen of the patient, it may be possible to process electrical potential vector signals corresponding to electrodes E1-E12 to determine a direction in which a muscle contraction travels across a patient's body over a period of time.

In some examples, electrical potential vector signals corresponding to electrodes E1-E12 may indicate one or more parameters of a muscle contraction other than direction. In some examples, electrical potential vector signals corresponding to electrodes E1-E12 may indicate a strength of a muscle contraction over a period of time. For example, when an electrical potential vector signal corresponding to electrode E3 is a first magnitude at a first time and the electrical potential vector signal corresponding to electrode E3 is a second magnitude at a second time, this may indicate that the muscle contraction decreased from the first time to the second time when the second magnitude is lower than the first magnitude. In some examples, electrical potential vector signals corresponding to electrodes E1-E12 may indicate an amount of time that a uterine contraction lasts, an amount of time between a uterine contraction and a subsequent uterine contraction, a variability of time intervals between uterine contractions, or any combination thereof.

It may be beneficial to process electrical potential vector signals corresponding to electrodes E1-E12 in order to generate information that assists clinicians in determining whether the patient is in labor. Since medical procedures must be performed when labor commences to successfully deliver the baby, a medical device system that differentiates true labor uterine contractions from non-labor uterine contractions may better prepare clinicians for providing care during pregnancy as compared with medical device systems that do not differentiate true labor uterine contractions from non-labor uterine contractions.

True labor uterine contractions occur when labor commences. True labor uterine contractions are strong uterine contractions that occur at regular time intervals and propagate distally, or downwards along longitudinal axis 170. In some examples, true labor uterine contraction travel in first direction 182. In some examples, the frequency of true labor uterine contractions increases as labor progresses. Non-labor uterine contractions such as Braxton-Hicks contractions may exhibit one or more characteristics that differentiate these non-labor uterine contractions from true labor uterine contractions. For example, Braxton-Hicks contractions may travel in directions that are not substantially downwards and parallel to longitudinal axis 170 (e.g., travel in second direction 184, third direction 186, or other directions). Braxton-Hicks contractions may, in some cases, be weaker than true labor uterine contractions. In some examples, Braxton-Hicks contractions may not occur according to predictable intervals like true labor uterine contractions do. In any case, a computing system may process biometric data collected by electrodes E1-E12 in order generate information that indicates whether true labor uterine contractions are occurring.

In some examples, FMS 300 may process biometric data collected by electrodes E1-E12 in order to determine a likelihood that the biometric data indicates that true labor uterine contractions are occurring in the patient during a period of time that the electrodes E1-E12 collect the biometric data. In some examples, FMS 300 may process biometric data collected by electrodes E1-E12 in order to determine a muscle contraction vector indicating the direction of a muscle contraction over the period of time. A muscle contraction vector may indicate the direction of the muscle contraction relative to longitudinal axis 170 over the period of time. In some examples, FMS 300 may process biometric data collected by electrodes E1-E12 in order to determine a timeseries identifying the strength of the muscle contraction over time.

In some examples, such as shown in FIG. 4A, wearable garment 164 extends entirely around the abdomen of the patient. In other examples, wearable garment 164 includes an opening or window on the back side of garment 164 to provide access to the lower back of the patient for purposes of administration of, for example, spinal or epidural anesthetic during labor and delivery, or for other reasons. In other examples, instead of a wearable garment 164, the sensors may be arrayed on a substrate and may include an adhesive that adheres the sensors and/or the substrate to the skin of the patient.

In accordance with one or more techniques of the disclosure, the physiological (e.g., biopotential) signals sensed by wearable device 151A and analyzed to determine the status of the fetus and/or predict one or more maternal and/or fetal outcomes may include, but are not limited to, fetal heart rate (fHR), maternal heart rate (mHR), fetal ECG, maternal ECG, and maternal EMG signals.

FIG. 4B is a diagram of a second example wearable device 151B including a first one or more sensors 152 (labeled E1-E12 and referred to generally as sensors 152 or electrodes 152) embedded or affixed to a wearable element 165 and a second one or more sensors 153 (labeled A1-A5, O1-O2, T1-T2 and referred to generally as sensors 153) embedded or affixed to a wearable element 165, in accordance with one or more techniques of the disclosure. Second example wearable device 151B may be an example of wearable device 150 of FIGS. 1A, 1B and 2. In some examples, wearable device 151B is configured for monitoring of one or more maternal and/or fetal physiological parameters during labor and delivery.

In this example, sensors 152 include a total of twelve electrodes E1-E12 affixed to or embedded within a wearable element 165 such that, when properly worn about the torso of a patient 181, the electrodes 152 are positioned about the torso of patient 181. One or more of the electrodes 152 may be positioned on the front, either side or back of the patient 181. As shown in FIG. 4B, in some examples, the sensor configuration on wearable element 165 is such that two electrodes are positioned on the back of patient 181 (E1 and E12 in this example). The number and configuration of sensors 152 on the wearable element 165 may vary from that shown in FIG. 4A, and the disclosure is not limited in this respect.

In some examples, electrodes 152 may include at least one reference electrode. For example, E11 may represent a reference electrode. A computing device (e.g., computing device 200) may, in some examples, analyze one or more signals generated by electrodes 152 in order to determine differences in electrical potential between one or more pairs of electrodes 152. For example, computing device 200 may determine a difference in electrical potential between E1 and E11, a difference in electrical potential between E2 and E11, a difference in electrical potential between E3 and E11, and so on. These determined differences in electrical potential may represent the ECG and/or EMG signals generated by sensors 152. In some examples, the reference electrode may be adaptively switchable as described below with respect to FIG. 4E.

In order to capture maternal and fetal biopotential signals of sufficient quality, sensors 152 should provide good contact with the patient's skin, minimize sensor movement relative to the skin, and reduce signal noise from light movements of the patient. Electrodes 152 may, in some examples, comprise “dry” electrodes that consist of a single metal which acts as a conductor between the skin and the electrode. In some examples, the dry electrodes may be attached to or detached from the wearable element 165.

In some examples, sensors 153 may include one or more acoustic sensors. In this example, sensors 153 include five acoustic sensors A1-A5, but this is not required. Other example wearable devices may include less than five acoustic sensors or more than five acoustic sensors. As seen in FIG. 4B, the acoustic sensors A1-A5 are spread out across the abdomen in order to sense a broad area. Each acoustic sensor of the acoustic sensors A1-A5 may measure an acoustic signal that indicates one or more sounds occurring in an area sensed by the respective sensor. In some examples, the acoustic signals measured by the acoustic sensors A1-A5 may indicate events such as maternal heart sounds, fetal heart sounds, maternal respiration sounds, blood flow, fetal movement, or any combination thereof. Computing device 200 may be configured to process the one or more acoustic signals in order to determine these events.

Since the acoustic signals may include maternal and/or fetal heart sounds, the acoustic signals may be useful for determining one or more aspects of maternal and/or fetal cardiac activity. For example, heart sounds may be indicative of a series of heart contractions, so the acoustic signals may indicate a heart rate of the mother and/or the fetus.

In some examples, when computing device 200 is monitoring signals from sensors 152, 153 in real-time, one or more signals received from electrodes 152 may cut off. For example, computing device 200 may stop receiving one or more signals from electrodes 152, or the signals received from electrodes 152 may decrease in quality to the point where computing device 200 can no longer adequately process the signals to determine cardiac activity. If one or more cardiac signals from electrodes 152 go offline, computing device 200 may turn to one or more signals from acoustic sensors A1-A5 in order to regain the lost cardiac signals. For example, computing device 200 may pivot from determining fetal heart rate and/or maternal heart rate based on signals from electrodes E1-E12 to determining fetal heart rate and/or maternal heart rate based on signals from acoustic sensors A1-A5. This may allow computing device 200 to deliver uninterrupted information to users even when one or more signals from electrodes E1-E12 go offline.

In some examples, sensors 153 may include one or more temperature sensors. In this example, sensors 153 include two temperature sensors T1-T2. This disclosure is not limited to wearable devices having two temperature sensors. It is possible that a wearable device may have more than two or less than two temperature sensors. Temperature sensors T1-T2 may, in some cases, generate one or more temperature signals that indicate a body temperature of the patient 181, a body temperature of the fetus, a temperature of an environment surrounding the patient 181, or any combination thereof. Computing device 200 may receive the one or more temperature signals, process the one or more temperature signals, make one or more determinations based on the temperature signals, output for display temperature information based on the one or more temperature signals, or any combination thereof.

In some examples, sensors 153 may include one or more optical sensors. In this example, sensors 153 include two optical sensors O1-O2. This disclosure is not limited to wearable devices having two optical sensors. It is possible that a wearable device may have more than two or less than two optical sensors. The optical sensors O1-O2 may measure one or more optical signals that indicate physiological parameters of patient 181. For example, optical sensors O1-O2 may measure one or more optical signals indicative of a blood pressure of patient 181.

In some examples, the one or more optical signals may indicate one or more pulse transit time (PTT) intervals. In some examples, computing device 200 is configured to measure one or more PTT intervals based on optical signals received from optical sensors O1-O2. A PTT interval may represent an amount of time between a ventricular depolarization of a heart of patient 181 and a subsequent peak or other feature of a signal corresponding to the contraction resulting from the R-wave. In some examples, optical sensors O1-O2 are configured to generate a photoplethysmography (PPG) signal indicative of a perfusion of blood to the dermis and subcutaneous tissue of patient 181. In this way, the PPG signal may represent a pulse of patient 181, where the PPG signal rises during a pulse and falls during periods between pulses. The PPG signal may reflect each heartbeat of patient 181 and/or the fetus, a PPG peak corresponding to a heartbeat.

In some examples, the optical sensors O1-O2 may include one or more pulse oximeters that are configured to generate optical signals indicative of pulse oximetry (SpO₂) values of the patient 181 and/or the fetus. In some examples, a PPG signal indicates SpO₂. SpO₂ may, in some cases, represent an approximation of arterial blood oxygen saturation (SaO₂). In some examples, SpO₂ and/or PPG signals may be indicative of a blood pressure of the patient and/or a blood pressure of the fetus.

In some examples, each of optical sensors O1-O2 may include one or more light emitters and one or more light detectors. Collectively, the one or more light emitters and the one or more light detectors may comprise an optical sensor, which may generate optical signals for determining SpO₂ values, PPG values, or color spectroscopy values as described herein. In some examples, each optical sensor the one or more optical sensors may be configured to emit light at one or more wavelengths using the light emitter(s) and receive light at one or more wavelengths using the light detector(s). Based on a frequency content of the emitted light and a frequency content of the received light, a computing device (e.g., computing device 200) may be configured to determine one or more parameters of an area in which the emitted light and the received light travelled. For example, a light emitter of an optical sensor may be configured to emit photons into the abdomen of the patient 181, and these photons may reflect off maternal tissue and/or fetal tissue and return to the light detectors of the optical sensor. The frequency content of photons returning to the light detectors may, in some cases, be affected by characteristics of one or more things that the photons reflected from. For

In some examples, a light emitter of an optical sensor may include a light source, such as a light-emitting diode (LED), that may emit light at one or more wavelengths within the visible (VIS) and/or near-infrared (NIR) spectra. In some examples, techniques for generating optical signals for determining SpO₂, PPG, or color spectroscopy may include using a light emitter to emit light at one or more VIS wavelengths and at one or more NIR wavelengths. The combination of VIS and NIR wavelengths may help enable processing circuitry of IMD 10 to distinguish oxygenated hemoglobin from deoxygenated hemoglobin, since as hemoglobin becomes less oxygenated, an attenuation of VIS light increases and an attenuation of NIR decreases. By comparing the amount of VIS light detected by light detectors to the amount of NIR light detected by light detectors, computing device 200 may determine the relative amounts of oxygenated and deoxygenated hemoglobin in the tissue. These relative amounts of hemoglobin may be indicative of PPG and/or SpO₂. For example, if the amount of oxygenated hemoglobin decreases, the amount of VIS light detected by the light detectors increases and the amount of NIR light detected by light detectors decreases. Similarly, if the amount of oxygenated hemoglobin in the tissue of patient 4 increases, the amount of VIS light detected by light detectors decreases and the amount of NIR light detected by light detectors increases.

Sensors 152, 153 may be integrated into the same system such that computing device 200 can process and analyze data received from sensors 152, 153 and determine whether to one or more actions based on the data received at any given time. As described above, computing device 200 may transition to using acoustic sensor data instead of data from electrodes 152 in response to one or more heart signals being lost in the data received from electrodes 152. Computing device 200 may evaluate signals received from each of sensors 152, 153 to determine a quality of these signals, and subsequently determine which signals to use in order to measure one or more maternal and/or fetal parameters at any given time. For example, computing device 200 may determine at a first time that one or more signals from electrodes E1-E12 are the most appropriate for determining fetal heart rate. At a second time, computing device 200 may determine that data from acoustic sensors and/or data from optical sensors is the most appropriate for determining fetal heart rate. Sometimes, computing determines that a combination of signals from different kinds of sensors can be used to determine fetal heart rate. But at any given time, computing device 200 may adjust in order to continuously deliver one or more maternal and/or fetal parameters in real time.

Electrodes E1-E12 may measure one or more signals that indicate contractions occurring in patient 181. There are several different kinds of contractions including standard labor contractions that occur during labor and delivery, and Braxton-Hicks contractions that prepare the uterine muscle for delivery. True labor uterine contractions are directional, starting at the fulcrum of the uterus and moving downwards. Braxton-Hicks contractions can occur in any direction. One or more techniques described herein include analyzing signals measured by sensors on the sensor array in order to determine the strength of one or more contractions and/or the direction of one or more contractions. This information may be important for determining whether a contraction is a true labor uterine contraction, a Braxton-Hicks contraction, or another kind of contraction.

In some examples, one or more processors of a medical device system (e.g., one or more processors of computing device 200 and/or FMS 300) may identify a true labor uterine contraction by analyzing the signals generated by electrodes E1-E12 and determining the direction of a contraction of the uterine muscle. True labor uterine contractions start at the fulcrum (the top) of the uterus and work their way downwards. When the signals from electrodes E1-E12 indicate that a contraction starts near E3 and E4, which are located proximate to the top of the uterus, the contraction subsequently progresses to E2, E5, E6, and E9, and the contraction subsequently progresses to E7, E8, and E10, the data may indicate a true labor uterine contraction. Computing device 200 may monitor a signal corresponding to each of electrodes E1-E12 and determine, based on a magnitude of each signal over time, when a contraction occurs and for how long. Computing device 200 may additionally or alternatively determine a strength of a contraction based on the data generated by electrodes E1-E12.

In some examples, one or more processors of a medical device system (e.g., one or more processors of computing device 200 and/or FMS 300) may identify a Braxton-Hicks contraction based on determining that the contraction moves in a direction other than from the top of the uterus to the bottom of the uterus. For example, when the data indicates that a contraction spreads from left to right, from right to left, from down to up, or in a diagonal direction, this may indicate that the contraction is likely a Braxton-Hicks contraction or another kind of contraction that is not a true labor uterine contraction. One or more processors of a medical device system (e.g., one or more processors of computing device 200 and/or FMS 300) may additionally or alternatively determine a strength and/or duration of a contraction and use this information to determine whether the contraction is a Braxton-Hicks contraction, a true labor uterine contraction, or another kind of contraction. Braxton-Hicks contractions may be less powerful than true labor uterine contractions.

In some examples, electrodes E1-E12 may be configured to output a set of electrical signals to one or more processors. In some examples, the set of electrical signals may define one or more electrical potential vector signals. Each electrical potential vector signals of the one or more electrical potential vector signals may represent a difference between an electrical signal corresponding to a reference electrode of electrodes E1-E12 and an electrical signal corresponding to another electrode of electrodes E1-E12. The one or more processors may, in some cases, be configured to process the electrical potential vector signals to generate information corresponding to one or more uterine contractions of patient 120.

For example, the one or more processors may be configured to determine, based on biometric data (e.g., electrical potential vector signals and/or other biometric data), a muscle contraction vector indicating a direction of the muscle contraction (e.g., a uterine contraction) over the period of time. In some examples, the muscle contraction vector may indicate a direction of a uterine contraction throughout a duration of the uterine contraction. The direction of a uterine contraction may be important for determining whether a uterine contraction is a true labor uterine contraction. For example, labor contractions may consistently travel in a downwards or distal direction substantially parallel to a longitudinal axis 170 of the patient. Therefore, generating the muscle contraction vector may provide a clinician with important information for determining whether a patient is in labor. In some examples, the one or more processors may execute a machine learning model to generate the muscle contraction vector. The machine learning model may accept biometric data such as electrical data collected by electrodes E1-E12 as an input, and generate the muscle contraction vector as an output.

In some examples, the one or more processors may be configured to determine, based on the biometric data, a likelihood that the muscle contraction comprises a true labor uterine contraction. The one or more processors may, in some cases, be configured to determine the likelihood in addition to or alternatively to determining a muscle contraction vector. In some examples, the one or more processors may execute a machine learning model in order to determine the likelihood that the muscle contraction comprises a true labor uterine contraction. The likelihood that the muscle contraction comprises a true labor uterine contraction may, in some cases, comprise a confidence score within a range from 0 to 1, with 0 being certain that the contraction is not a true labor uterine contraction, and 1 being certain that the contraction is a true labor uterine contraction. In some examples, the one or more processors may be configured to determine, based on the biometric data, a timeseries that indicates a strength of a contraction over a period of time.

Wearable element 165 may be configured to contact and/or attach to the patient 181 such that sensors 152, 153 can adequately collect data. Wearable element 165 may, in some cases, represent a wearable textile garment that extends around a body of a patient 181, but this is not required. Wearable element 165 may comprise one or more “patches” that attach sensors 152, 153 to the skin of the patient 181 using adhesive. In some examples, wearable element 165 may comprise a mechanical apparatus that attaches the sensors 152, 153 to the patient 181. This mechanical apparatus may, in some cases, include one or more adjustable legs that allow for one or more sensors to be brought flush to the skin and allow one or more sensors to be separated from the skin at the discretion of the user.

FIG. 4C is a diagram of a third example wearable device 151C including a first one or more sensors 152 (labeled E1-E16 and referred to generally as sensors 152 or electrodes 152) embedded or affixed to a wearable element 165 and a second one or more sensors 153 (labeled A1-A5, O1-O2, T1-T2 and referred to generally as sensors 153), in accordance with one or more techniques of the disclosure. Third example wearable device 151C may be an example of wearable device 150 of FIGS. 1-2.

In some examples, third example wearable device 151C of FIG. 4C may be substantially the same as second example wearable device 151B of FIG. 4B, except that third example wearable device 151C includes additional electrodes E13-E16. As seen in FIG. 4C, electrodes E13 and E14 are located proximate to gluteal muscles of the patient 181, and electrodes E15 and E16 are located proximate to groin muscles of the patient 181. The techniques described herein are not limited to two electrodes proximate to the gluteal muscles and two electrodes located proximate to the groin muscles. In some examples, there may be less than two electrodes or more than two electrodes proximate to the gluteal muscles and there may be less than two electrodes or more than two electrodes proximate to the groin muscles.

In some examples, electrodes E1-E16 may generate one or more signals that indicate muscle activity. For example, signals generated by electrodes E1-E6 may indicate which muscles are contracting during labor. In some cases, clinicians may provide feedback to a patient during delivery in order to help the patient through the delivery. This feedback may include instructions for how to apply certain muscles in order to push the baby out of the uterus. It may be beneficial for the clinician to view signal data that indicates muscles one or more muscles used, and for the clinician to instruct the patient on how to use the correct muscles if the patient is not applying the correct muscles.

Electrodes E1-E16 may generate one or more signals that indicate one or more contractions occurring in patient 120. There are several different kinds of contractions including standard labor contractions that occur during labor and delivery, and Braxton-Hicks contractions that prepare the uterine muscle for delivery. True labor uterine contractions are directional, starting at the fulcrum of the uterus and moving downwards. Braxton-Hicks contractions can occur in any direction. One or more techniques described herein include analyzing signals generated by sensors on the sensor array in order to determine the strength of one or more contractions and/or the direction of one or more contractions. This information may be important for determining whether a contraction is a true labor uterine contraction, a Braxton-Hicks contraction, or another kind of contraction.

FIG. 4D is a conceptual diagram illustrating a cross-section view of the abdomen of patient 181 and the wearable element 165, in accordance with one or more techniques of this disclosure. As seen in FIG. 4D, the wearable element 165 may rest on top of the abdomen of the patient 181. In some examples, adhesive may attach the wearable element 165 to the patient 181. In some examples, one or more mechanical elements may attach the wearable element 165 to the patient 181. Wearable element 165 might, in some cases, not extend all the way around a circumference of the patient's body. Wearable element 165 might attach to a portion of the abdomen such that sensors can sense one or more signals indicative of fetal and maternal parameters.

FIG. 4E is a more detailed block diagram of the electronic components of an example wearable device 700, in accordance with one or more techniques of the disclosure. In some examples, wearable device 700 may be used to implement wearable device 150 and/or 150B of FIGS. 1A, 1B, and 2. Example wearable device 700 includes one or more first sensors 752A-752N, one or more second sensors 753A-753N, a mount 718, and a “core” 754 including the control electronics for wearable device 700. In this example, core 754 is configured to be removably connected to a wearable garment or band (such as shown in FIG. 4A) via a mount 718. However, in some examples, wearable device need not include a core configured to be removably connected to the wearable garment or band, and the disclosure is not limited in this respect. First sensors 752A-752N may include sensors for sensing one or more biopotential (e.g., ECG, EMG, etc.) signals. Second sensors 753A-753N may include sensors for sensing other physiological parameters including acoustic sensors, temperature sensors, optical sensors, etc.

In this example, core 754 includes reference selector circuitry 712, analog front end circuitry 714, a communication interface or radio 758, a power source 764 (e.g., one or more batteries), one or more processor(s) 722, and/or storage device(s) 724. Storage device(s) 724 are configured to store executable control instructions and/or raw sensor data associated with one or more monitoring sessions. In some examples, wearable device 700 may store sensor data temporarily during each monitoring session for wireless transmission to a computing device, or wearable device 700 may store sensor data associated with multiple monitoring sessions for later transmission to a computing device.

During an initialization period, the computing device obtains maternal and/or fetal biopotential signal data for a patient from each of a plurality of electrode pairs selected from among electrodes 752A-752N. One electrode of each electrode pair may be referred to as a reference electrode and the other electrode of each electrode pair may be referred to as a measurement electrode. According to one or more techniques of the disclosure, the reference electrode is adaptively switchable between two or more of the plurality of electrodes. For example, the total number of electrode pairs is given by the following equation:

$$Total Number of Possible Electrode Pairs = \frac{N !}{2 ( N - 2 ) !}$$

As one example, if n=11 (e.g., the wearable device includes 11 electrodes which may be used to measure the maternal/fetal biopotential signal(s), such as E1-E11 as shown in FIG. 4A) there are 55 different reference-measurement electrode pair combinations.

For example, if the computing device determines that electrode E2 should be selected as the reference electrode during an initialization period, the following electrode pairs are used to acquire the maternal and/or fetal biopotential signals during a measurement period of a monitoring session:

### Pair 1: E1 to E2

### Pair 2: E3 to E2

### Pair 3: E4 to E2

### Pair 4: E5 to E2

### Pair 5: E6 to E2

### Pair 6: E7 to E2

### Pair 7: E8 to E2

### Pair 8: E9 to E2

### Pair 9: E10 to E2

### Pair 10: E11 to E2

Reference selector circuitry 712 is used to select one of the plurality of electrodes to use as the reference electrode during the measurement period of the monitoring session. Processor(s) 722 may receive instructions from the FMS 300 indicating which electrode 752A-752N should be used as the reference electrode for the current monitoring session, and controls reference selector 712 to select the correct reference electrode and to receive maternal and/or fetal biopotential signals from each of the electrode pairs formed by the reference electrode and each one of the remaining electrodes. Analog front end includes one or more amplifiers which are used to amplify the maternal and/or fetal biopotential signal from the selected electrode pair, and an analog to digital converter (ADC). The digitized signal(s) are wirelessly communicated by a radio 758 (e.g., communication interface 158 as shown in FIG. 2) for receipt by a patient computing device (e.g., patient computing device 200 as shown in FIG. 2) and/or by a LD computing system 190 as shown in FIG. 1B.

In accordance with one or more techniques of the disclosure, in some examples, wearable device 700 may further implement a noise reduction approach for mixed-signal monitoring by using one of the electrodes 752A-752N as a driven electrode. The purpose of the driven electrode is to provide low current, low voltage potential to bias the body to the same potential and reduce the impact of bias offset at each electrode measuring location. The usage of a driven electrode to provide a signal to bias the body may reduce the noise in the maternal and/or fetal biopotential signal data collected by the remaining sensors of the wearable device by cancelling out common mode interference in the sensed maternal and/or fetal biopotential or other physiological signals. The usage of a driven electrode may thus result in higher quality maternal and/or fetal biopotential or other physiological signals for ongoing monitoring or analysis purposes. In examples where one of the electrodes is a driven electrode, the driven electrode is not used for measurement of the maternal and/or fetal biopotential signals.

For example, one of electrodes 752A-752N may be selected as the driven electrode. During a monitoring session, driven electrode is supplied with a low current, low voltage potential signal to bias the body to the same potential and reduce the impact of bias offset at each electrode measuring location. In this way, each of the remaining electrodes 752A-752N are biased to the same potential regardless of their location on the body of the patient. This may help to cancel out common mode interference in the sensed maternal and/or fetal biopotential signal sensed by the remaining electrodes, resulting in a higher quality biopotential (e.g., ECG, EMG, etc.) signal and more accurate determination of fetal heart rate and generation of one or more predicted outcomes.

In some examples, the driven electrode may be adaptively selectable in a manner similar to that described above with respect to the reference electrode. For example, a computing device (such as any one of FMS 300, patient computing device 200, or provider computing system 180) may be configured to select, based on processing the maternal and/or fetal biopotential signal data sensed by each of the plurality of electrode pairs, one of the plurality of electrodes to use as the driven electrode during the measurement period of the monitoring session for the patient.

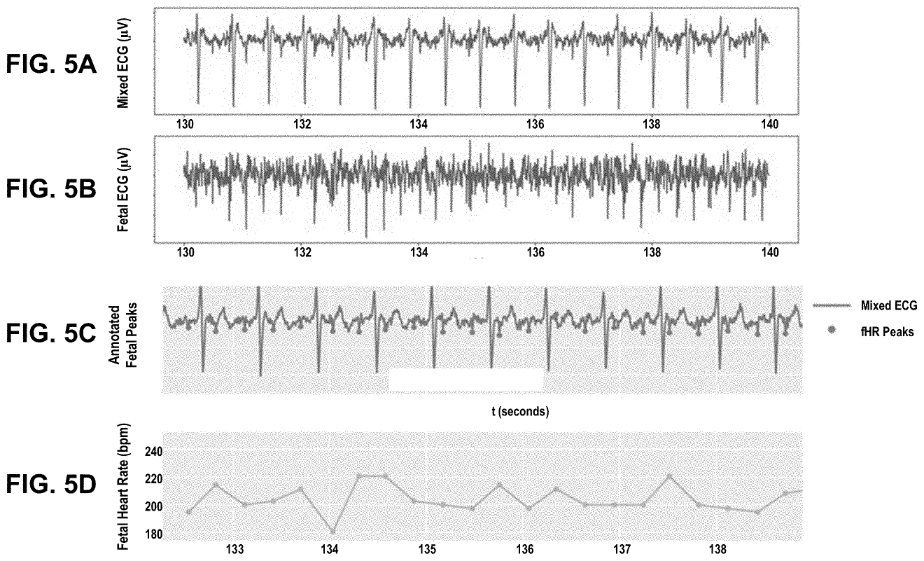

FIGS. 5A-5D are graphs illustrating an example mixed maternal-fetal ECG signal (FIG. 5A), a fetal ECG signal extracted from the mixed maternal-fetal ECG signal (FIG. 5B), a graph showing an identification of peaks in the fetal heart rate (FIG. 5C), and a presentation of fetal heart rate as determined based on the fetal ECG signal (FIG. 5D), respectively, in accordance with one or more techniques of the disclosure.

To obtain the fetal ECG (FIG. 5B) from the mixed (maternal and fetal) ECG signal (FIG. 5A) that is captured by the wearable device, an extraction algorithm may be employed. Example techniques for extracting a fetal ECG signal (FIG. 5B) from a mixed maternal-fetal ECG signal are described in United States Patent Application Publication No. 2020/0113470, published on Apr. 16, 2020, which is incorporated by reference herein in its entirety.

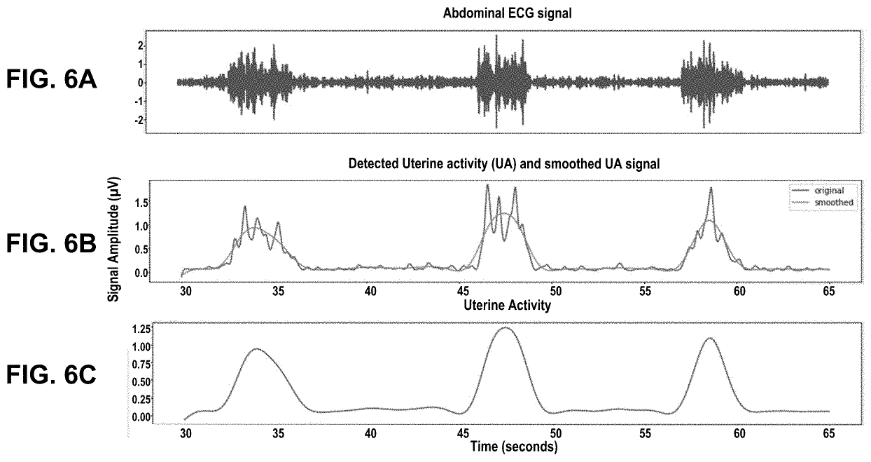

FIGS. 6A-6C are graphs showing an example abdominal (e.g., EMG) signal obtained using the example wearable device 150 as shown in FIG. 4A (FIG. 6A), the envelope of the original uterine activity (UA) and smoothed UA signal (FIG. 6B) and the detected uterine activity signal (FIG. 6C), in accordance with one or more techniques of the disclosure.

Any one or more of the graphs shown in FIGS. 5A-5D and/or 6A-6D may be displayed on a user interface of LD computing system 190 such as that shown in FIG. 1B.

FIG. 6D is a combined fetal heart rate and uterine signal graph in accordance with one or more techniques of the disclosure. Example features of the fetal heart rate signal (upper portion of the graph) are indicated by reference numerals 172 and 174 and example features of the uterine contraction signal (lower portion of the graph) are identified by reference numerals 176 and 178. Reference numeral 172 indicates an acceleration of the fetal heart rate signal that occurred during a first period of time. Reference numeral 174 indicates a deceleration of the fetal heart rate signal that occurred during a second period of time. The detected accelerations/decelerations of the fetal heart rate signal (or the average, mean or other statistical characterization of the detected accelerations/decelerations) may be extracted as a feature that is input to the ML models for the prediction of maternal and/or fetal outcomes. Reference numeral 176 indicates the start of a uterine contraction substantially corresponding to the first period of time during which the fetal heart acceleration indicated by reference numeral 172 occurred. Reference numeral 178 indicates the end of the uterine contraction substantially corresponding to the second period of time during which the fetal heart rate deceleration 174 occurred.

One or more features of the sensed biopotential signals may be extracted and used as inputs to a machine learning model (such as ML model(s) 310) to predict one or more maternal and/or fetal outcomes. For example, features of the fetal heart rate may include baseline heart rate, baseline variability, number of accelerations per second, number of early, late, and variable decelerations per second, number of prolonged decelerations per second, sinusoidal pattern, etc. Features of the fetal ECG may include, for example, one or more features of the P wave, QRS complex, T wave, PQ interval, QRS duration, QT interval, RR interval, or other feature indicative of the electrical activity of the heart (e.g., start, end, duration, amplitude, peak-to-peak information, morphology, etc.). In another example, analysis of the raw fetal ECG signal may be considered as well to avoid the information loss associated with such feature extraction procedures.

Similar features may also be identified for the maternal heart rate. Uterine contraction (UC) features may include baseline uterine tone, contraction frequency, start/end time of uterine contractions, amplitude of uterine contractions, duration of uterine contractions, and strength (intensity) of uterine contractions.

Example features of the fetal heart rate may include, but are not limited to, the features shown in Table 1. Similar features may also be identified with respect to the maternal heart rate.

TABLE 1
Variable Description (fHR)

Fetal heart rate baseline (beats per minute)
Number of accelerations
Number of fetal movements
Number of uterine contractions
Number of moderate decelerations
Number of severe decelerations
Number of prolonged decelerations
Percentage of time with abnormal short-term variability
Mean duration of short-term variability
Mean duration of long-term variability
Percentage of time with abnormal long-term variability
Histogram tendency
Fetal state class code (N = Normal, S = Suspected, P = Pathological)
Width of FHR histogram
Minimum of FHR histogram
Maximum of FHR histogram
Number of histogram peaks
Number of histogram zeroes
Histogram mode
Histogram median
Histogram variance
Amplitude of FHR

The patient data for a particular patient may include patient data obtained during one or more previous monitoring sessions for the patient. The patient data associated with the previous monitoring sessions may thus be used to establish one or more baselines for the patient. For example, baselines with respect to maternal ECG and/or heart rate, fetal ECG and/or heart rate, etc., may be established and used as feature inputs to one or more ML models for prediction of maternal and/or fetal outcomes for the patient. In this way, longitudinal information for the patient over time may be taken into account when determining the one or more maternal and/or fetal outcome predictions for the patient.

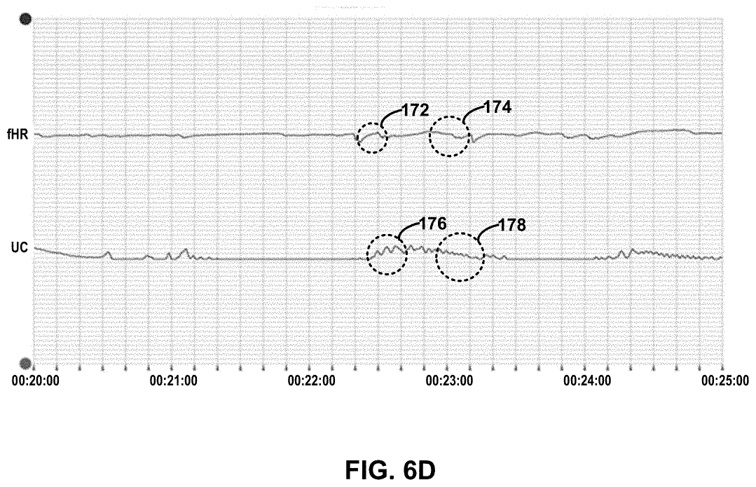

FIG. 7 is a conceptual diagram illustrating an example of training and using a machine learning model that predicts one or more outcomes and/or generates uterine contraction information, in accordance with one or more techniques of the disclosure. The conceptual diagram of FIG. 7 includes AI engine 302 and ML model(s) 310 as shown in FIGS. 1 and 3 and illustrates one example of training and using ML model(s) 310 to predict one or more fetal and/or maternal outcomes and generate uterine contraction information. In some examples, AI engine 302 is configured to use supervised or unsupervised machine learning techniques to train one or more ML model(s) 310 to predict one or more fetal or maternal outcomes 380. In some examples, AI engine 302 is configured to use supervised or unsupervised machine learning techniques to train one or more ML model(s) 310 to generate uterine contraction information 382. The techniques of the disclosure result in optimized predictive analytics that detect and classify the patient data received from the wearable device (and/or other patient data such as biometric data, health assessment data, socio-economic data, etc.) using historical data from a plurality of patients, to result in one or more ML models that provide detailed insights into the health of prenatal and postpartum patients, or during labor and delivery.

During a training phase, AI engine 302 receives training data 344 that includes, for example, historical patient data associated with a plurality of patients and/or muscle contraction data associated with a plurality of patients. Training data 344 for a particular patient may include, for instance, values of the maternal and/or fetal ECG or heart rate data obtained during one or more monitoring sessions. The training data may include the raw heart rate trends, pressure measurements as they relate to contractions, and the matched fetal outcomes, maternal outcomes, and data about the subjects. The training data may also include umbilical cord arterial and venous pH levels for hypoxia detection. Additionally, or alternatively, the training data may include biometric data associated with one or more different kinds of uterine contractions (e.g., true labor uterine contractions, Braxton-Hicks contractions, or other kinds of uterine contractions).

One or more features 350A-350N may be extracted from the training data. The features may include independent and dependent variables. For example, the features may include any one or more features of the fetal ECG, the maternal ECG, the fetal heart rate, the maternal heart rate, and/or the uterine contractions (e.g., EMG). The features may also include features of other biometric data including blood pressure, weight, glucose, pH blood levels, blood oxygen level, breathing rate, patient movement, temperature, etc., feature of one or more mental health assessments, social determinates of health (SDoH) assessments, socio-economic data, and any other data that may be relevant to a determination of fetal or maternal outcomes.

In some examples, before the modelling takes place, the following features may be extracted from the raw data and used as one or more features of the training data: distribution of gestation week at delivery, distribution of fHR signal duration, percentage of records with validated cord blood gas analysis and pH level for those records, distribution of all fetal outcomes of interest in this study.

Features 350A-350N may be selected manually, for example, by a subject matter expert or automatically, for example, by a feature extractor that is part of AI engine 302. A feature extractor may also be used to indicate feature importance or weights for each of the features. Feature importance can be used to determine the relative importance of each feature with respect to the strength of the association of that feature in predicting each of the one or more outcomes 380 or generating the uterine contraction information 382. The set of features may be refined by performing mathematical, statistical, and heuristic procedures to identify an optimal set of inputs to AI engine 302. In some examples, one or more of the features may include one or more known maternal and/or fetal outcomes corresponding to the historical patient data for each of a plurality of patients. In some examples, one or more of the features may include features associated with true labor uterine contractions and features associated with non-labor contractions. For example, features associated with true labor uterine contractions may include a direction, a strength, and a frequency and features associated with non-labor contractions

In some examples, according to one or more techniques of the disclosure, AI engine 302 applies a training data set 350A-350N including patient data and associated outcomes obtained for each of a plurality of patients to train one or more ML model(s) 310 to predict one or more maternal and/or fetal outcomes. The ML model(s) are indicative of which features of the patient data are predictive of one or more maternal or fetal outcomes (either adverse or non-adverse). In some examples, according to one or more techniques of the disclosure, AI engine 302 applies a training data set 350A-350N including uterine contraction data for each of a plurality of patients to train one or more ML model(s) 310 to generate uterine contraction information.

During a deployment phase, the trained ML model(s) 310 may be deployed for use by AI engine 302 to predict one or more fetal and/or maternal outcomes 380 and/or generate uterine contraction information 382 for a particular patient based on current data 346 acquired during a monitoring session or health assessment session for the particular patient. During operation, one or more features 350A-350N are extracted from current data 346 acquired by a wearable device (such as wearable device 150) during the current monitoring session or health assessment session (such as via patient computing device 200 executing patient application 208). AI engine 302 processes the features 350A-350N of the current data 346 using machine learning model 310 to generate one or more predicted outcomes 380. In some examples and as shown in FIG. 7, the one or more features 350A-350N received by AI engine 302 during the current session including one or more of the same features 350A-350N of the training data 344 that were used to train machine learning model 310.

The predicted outcome(s) 380 may be expressed in various ways. For example, the predicted outcome(s) 380 may include predicted future value(s) of one or more fetal and/or maternal biometric or physiological parameter(s). The predicted outcome(s) 380 may include one or more predicted outcome classification(s). The predicted outcome(s) 380 may include a probability that one or more predicted outcomes (e.g., either adverse or non-adverse) will occur at some time in the future. The predicted outcome(s) 380 may further include any one or more of a confidence interval, a confidence level, etc.

The uterine contraction information 382 may, in some examples, include a likelihood that one or more uterine contractions of the patient represent true labor uterine contractions. Additionally, or alternatively, the uterine contraction information 382 may include a muscle contraction vector indicating a direction of a uterine contraction over a period of time. Additionally, or alternatively, the uterine contraction information 382 may include a timeseries indicating a strength of a uterine contraction over a period of time.

In some examples, the training data 344 includes maternal/fetal ECG and fetal heart rate data and associated outcomes obtained for each of a plurality of patients (e.g., pregnant human mothers and their fetuses), and is used to train and validate machine learning model 310 for maternal and/or fetal outcome prediction. The machine learning methods are used to determine which maternal and/or fetal ECG or heart rate patterns (e.g., fetal heart rate variability patterns or features) are predictive of adverse and/or non-adverse outcomes. The model may be tested on portion of the training dataset or on different data sets to compare approaches with the goal of developing ML model(s) 310 that outperform current methods to find patterns related to outcomes imperceptible to human interpretation. The maternal/fetal ECG and/or heart rate measurements together with the recorded outcomes as well as the patterns determined may be used as training data. The dataset may be split into a development and a holdout/test data set. The development data set may then be further divided into training and internal validation data, then used in real-time for the assessment of fetal heart rate data (such as current data 346) to predict one more or more outcomes 380. Hyperparameters such as the batch size, the initial learning rate, the number of neurons in the fully connected layers, and the number of convolutional layers may be adjusted to obtain an optimal model based on the validation set. New maternal and/or fetal heart rate data outside of the original training dataset(s) may be used to update or continuously update the machine learning model. This AI-driven assessment enables continuous improvement of the system and accuracy of the predicted outcomes.

The techniques of the disclosure thus identify features of, for example, maternal and/or fetal heart rate that may help decrease the incidence of adverse perinatal outcomes, including fetal acidemia, fetal hypoxia, and births by Cesarean section (C-section). In this way, the techniques of the disclosure may lead to early intervention intended to address or reduce the impact of predicted adverse events, resulting in improved maternal and fetal outcomes and decreased costs associated with adverse outcomes. This may help to maximize clinical effectiveness and speed to which a clinical team can react to clinical situations where there may be a need for intervention to help a mother and her unborn baby.

Maternal and/or fetal heart rate data can be captured over time using various technologies including, but not limited to, CTG (cardiotocography), fetal scalp electrodes, electrodes that capture fetal ECG (fetal electrocardiogram), acoustic sensors, etc. In some examples, the maternal and/or fetal heart rate data is captured by one or more sensors for capturing maternal/fetal ECG and EMG (contractions) signals incorporated into a wearable device such as wearable device 150 as shown in FIGS. 1 and 2.

In accordance with one or more techniques of the disclosure, the system may determine, based on processing patient data associated with a patient using one or more ML model(s) trained with historical patient data, one or more predicted outcomes associated with the patient (such as predicted outcomes 380). The predicted outcomes may include, but are not limited to:

Fetal outcomes:
Apgar scores (1, 5 and 10 minutes after birth)
Cord blood gas pH level
Neonatal destination immediately after birth
Admission to Neonatal Intensive Care Unit (NICU) within 48 hours of birth
NICU length of stay
Resuscitation intervention
Other neonatal complications
Additional adverse fetal outcomes (e.g., growth restriction, reduced fetal movement, delayed or absent cardiac response to fetal movement and contractions, atrial fibrillation, arrhythmia, brady/tachy syndrome, etc.)
Respiratory adverse outcomes (central, obstructive, mixed apnea, hypopnea, etc.)
Neonatal death up to 28 days after birth

Maternal outcomes:
Mode of delivery—vaginal or C-section
Reason for C-section
Grade of C-section (If performed—Grades 1, 2, 3 or 4)
Length of stay
Destination immediately after birth
Admission to a higher level of care
Complications (type and severity)
Additional adverse outcomes (e.g., preeclampsia, eclampsia, gestational hypertension, gestational diabetes, etc.)
Additional data may include:
Hour of day of delivery
Day of week of delivery

In some examples, in addition or alternatively to fetal ECG/fetal heart rate, maternal ECG/maternal heart rate, and uterine contraction data, the training data 344 and/or current data 346 may include any one or more of blood pressure, weight, glucose, pH blood levels, blood oxygen level, breathing rate, patient movement, temperature, mental health assessments, social determinates of health (SDoH) assessment, other data linked to clinical data, and/or any other biometric data or data relevant to prediction of maternal and/or fetal outcomes. This training data may be used to generate the ML models for the identification of high-risk pregnancies (e.g., prediction of one or more adverse outcomes described herein). By including additional parameters, false predictions of fetal distress that may lead to unnecessary Cesarean sections may be minimized. At the same time, accuracy regarding the prediction of actual fetal distress may be maximized, allowing for timely interventions when needed. The techniques of the disclosure thus provide a comprehensive and accurate monitoring system that takes many attributes, features, and/or patterns of fetal and/or maternal heart rate into account when predicting one or more maternal and/or fetal outcomes.

Different machine learning classification models may be trained using the training data. Sensitivity, precision, and F1 score for each class and overall accuracy of each model may be obtained to predict normal, suspect, and pathological states. The ML model with the best performance on specified metrics will be then identified and reported for each identified outcome, The ML model(s) are stored as ML models 310 and used for prediction of fetal and/or maternal outcomes 380 during a current monitoring session or based on previously monitored data acquired during a session of interest.

For example, a first ML model may be trained to predict a preterm labor risk. A second ML model may be trained to predict a preeclampsia risk. A third ML model may be trained to predict a C-section risk related to preeclampsia, diabetes, and/or body mass index (BMI). A fourth ML model may be trained to predict “high-risk” pregnancies. Similarly, one or more additional ML models may be trained to predict one or more outcomes. In this way, multiple ML models may be generated, each associated with one or more adverse or non-adverse outcomes. The ML models may be stored (e.g., as ML model(s) 310 as shown in FIGS. 1 and 2 and/or ML model 310 as shown in FIG. 7) and applied to patient data obtained during a current monitoring session (or applied to patient data acquired during a previously conducted monitoring session) to predict one or more adverse or non-adverse outcomes.

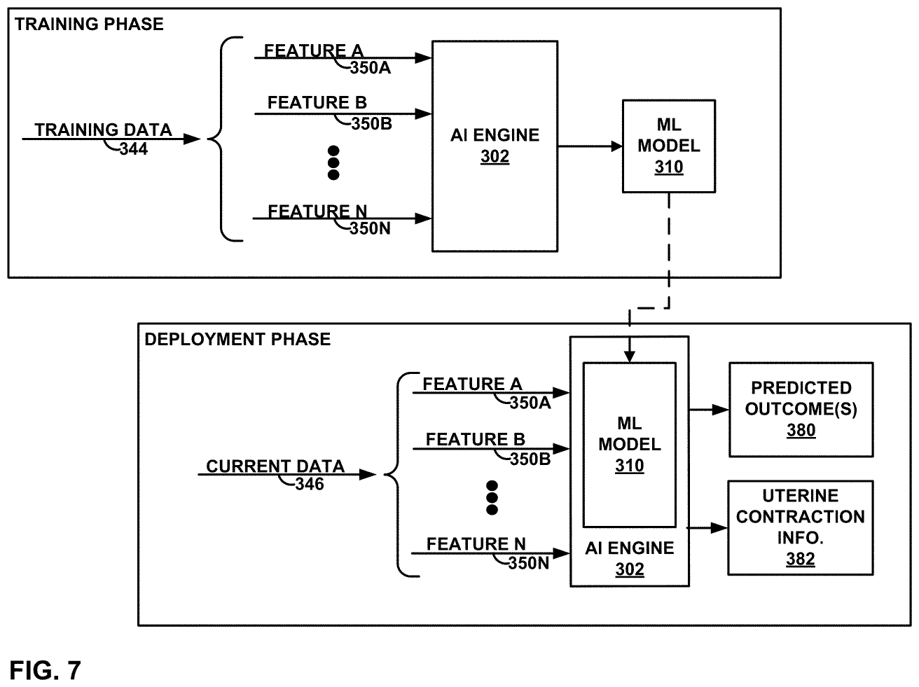

In some examples, the techniques of the disclosure develop prediction models using any one or more of generalized or specialized machine learning applications. These may include, for example, any one or more of random forest (RF), RBF kernel SVM, linear SVM, linear regression and/or logistic regression techniques. The machine learning techniques may further include any one or more of deep multilayer perceptrons (MLP), convolutional or deep convolutional neural networks (CNN), recurrent neural networks (RNN), long short-term memory neural networks (LSTM), artificial neural network (ANN), deep belief networks (DBN), Bayesian networks, autoregressive models, fuzzy-logic systems, hidden Markov models (HMM), Gaussian process models, etc. In one example, the techniques of the disclosure develop prediction models using a Convolutional Neural Network (CNN) and/or a Recurrent Neural Network (RNN) approach based on the Keras Framework with a Tensorflow (Google, Mountain View, CA) backend. In the example of a CNN, a CNN consists of an input and an output layer, as well as multiple hidden layers. Hidden layers of a CNN typically include convolutional layers, pooling layers and fully connected layers that are used to extract features. During training of a CNN, for example, the weights of the convolutional filters may be adjusted to extract meaningful and relevant features in an unsupervised way, and each task may be defined by the outcome to be predicted. FIG. 8 shows an example visualization of a convolutional neural network (CNN) for classification (e.g., prediction) using ECG signal data.

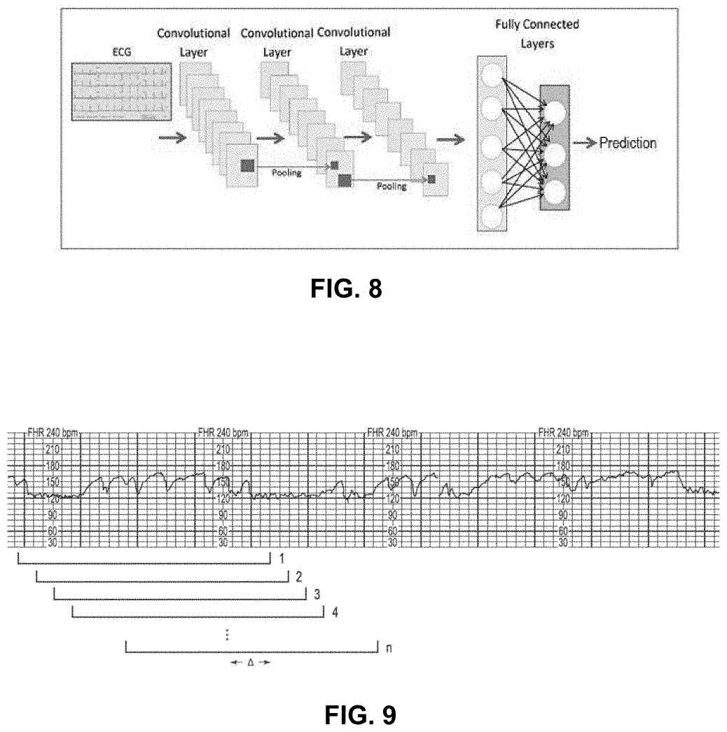

In some examples, before continuous data, such as fetal heart rate recordings, are used as input for any of the techniques described above, they undergo a segmentation procedure. Each recording is randomly partitioned in n different ways into recordings of length A. FIG. 9 shows an example segmentation batch using a thumbing window. The exact sizes of Δ and n are subject to optimization during the project. It may also be necessary to develop a “smart” way of segmenting recordings if the randomized approach does not deliver satisfactory results (in relation to its computational cost).

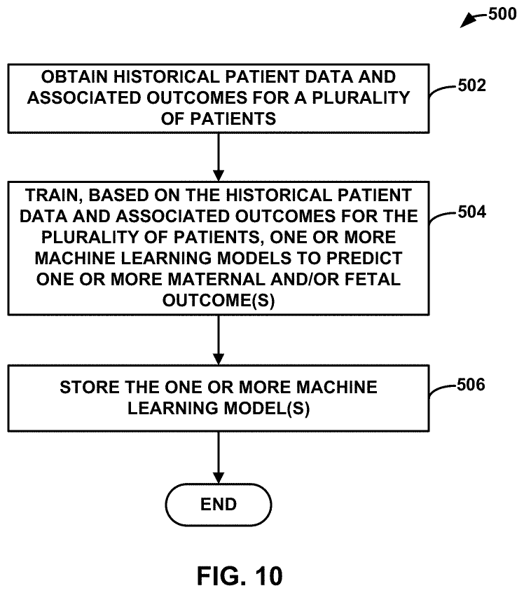

FIG. 10 is a flow chart illustrating an example process (550) by which a computing device, such as one or more processor(s) 302 of FMS 300, may train one or more machine learning models to generate one or more maternal and/or fetal outcome predictions in accordance with one or more techniques of the disclosure. The computing device obtains patient data and associated outcomes for a plurality of patients (502). The computing device trains, based on the historical patient data and associated outcomes for a plurality of patients, one or more machine learning models to predict one or more maternal and/or fetal outcomes (504). The computing device stores, for example, as ML models 310 as shown in FIGS. 1 and 3, the one or more machine learning models for later prediction of one or more maternal and/or fetal outcomes (506).

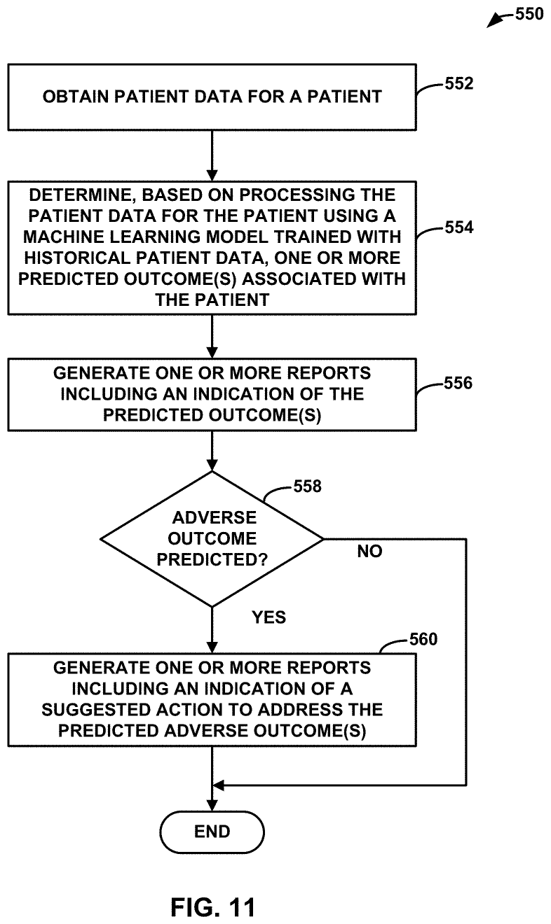

FIG. 11 is a flow chart illustrating an example process (550) by which a computing device, such as one or more processor(s) 302 of FMS 300, may generate one or more maternal and/or fetal outcome predictions in accordance with one or more techniques of the disclosure. The computing device obtains patient data for a patient acquired during a patient monitoring session (552). The computing device determines, based on processing the patient data for the patient using a machine learning model trained with historical patient data for a plurality of patients, one or more predicted maternal and/or fetal outcomes associated with the patient (554). The computing device may generate one or more reports including an indication of the predicted outcomes for display on one or more computing devices (558). For example, the computing device may execute a patient module (such as patient module 306) or a provider module (such as provider module 308) to generate the one or more reports for display on a patient computing device or a provider computing system, respectively.

The computing device may further determine if the predicted outcome is an adverse outcome (558). If the predicted outcome is not an adverse outcome (NO branch of 558), the process of predicting one or more maternal or fetal outcomes for the monitoring session is complete. If the predicted outcome is an adverse outcome (YES branch of 558), the computing device may generate one or more reports including an indication of a suggested action or actions that may be taken to address the predicted adverse outcome(s) (560). For example, the suggested actions for a patient may include a suggestion that the patient change one or more habits, a suggestion that the patient contact their healthcare provider, a suggestion that the patient change the frequency of their monitoring sessions, etc. As another example, the suggested actions for a provider may include a diagnostic suggestion, an intervention suggestion, a care plan suggestion, etc.

As one specific example of process (550) of FIG. 10, a computing device, such as one or more processor(s) 302 of FMS 300, may train one or more machine learning models to generate a predicted increase in fetal pH blood levels, in accordance with one or more techniques of the disclosure. The computing device obtains historical maternal and/or fetal ECG or heart rate data and associated known outcomes for a plurality of patients (502). The associated known outcomes include an increase in fetal pH blood levels for a plurality of patients. The computing device trains, based on the historical maternal and/or fetal ECG or heart rate data and associated known outcomes including increases in fetal pH blood levels for the plurality of patients, one or more machine learning models to predict one or more maternal and/or fetal outcomes (504). The computing device stores, for example, as ML models 310 as shown in FIGS. 1 and 3, the one or more machine learning models for later prediction of one or more maternal and/or fetal outcomes, including the prediction of the increase in fetal pH blood levels (506).

In some examples, certain features of the fetal heart rate satisfying respective threshold(s) may be correlated to an increase in fetal pH blood levels and thus processed by the associated ML model to predict an increase in fetal pH blood levels. For example, an increase in the fetal heart rate satisfying one or more thresholds may be determined during training of the ML model to be correlated to a predicted increase in fetal pH blood levels. The one or more thresholds may include, for example, a specified increase/decrease in the frequency of the fetal heart rate, a specified increase/decrease in the amplitude of the fetal heart rate, a specified increase/decrease in the frequency of the fetal ECG signal, a specified increase/decrease in the amplitude of the fetal ECG signal, etc. In addition, a prediction of an increase in fetal pH blood levels may be further correlated to a predicted risk of preeclampsia and/or C-section outcomes. Conversely, training of the one or more ML models may reveal that certain features or combinations of features are indicative that a C-section is not indicated. In such cases, unnecessary C-sections and associated medical costs, along with increased recovery time for the mother, may be avoided.

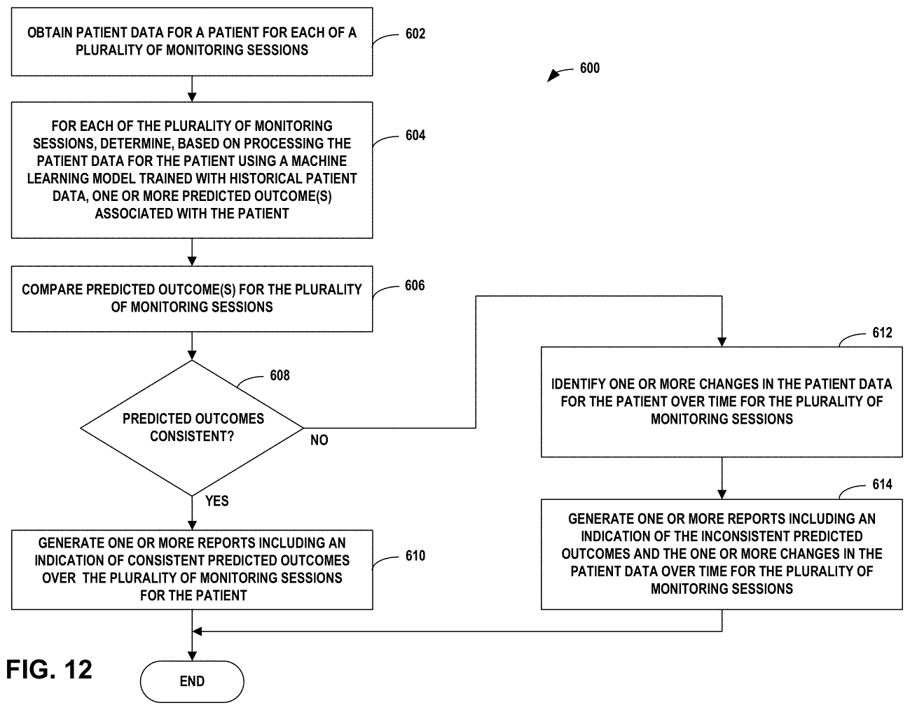

FIG. 12 is a flow chart illustrating an example process (600) by which a computing device, such as one or more processor(s) 302 of FMS 300, may generate one or more maternal and/or fetal outcome predictions based on longitudinal tracking of patient data for a particular patient in accordance with one or more techniques of the disclosure. The computing device obtains patient data for a patient acquired for each of a plurality of patient monitoring sessions (602). For each of the plurality of monitoring sessions, the computing device determines, based on processing the patient data for the patient using a machine learning model trained with historical maternal and/or fetal ECG or heart rate data for a plurality of patients, one or more predicted maternal and/or fetal outcomes associated with the patient (604). The computing device compares the one or more predicted outcomes determined during each of the plurality of monitoring sessions with the one or more predicted outcomes determined during the remaining plurality of monitoring sessions (606). The purpose of the comparison is to monitor the predicted outcomes for a patient longitudinally over time (e.g., over a plurality of monitoring sessions) to determine whether each predicted outcome is consistent with one or more previous or subsequent predicted outcomes (e.g., predicted outcomes determined for a previous or subsequent monitoring session). If the predicted outcomes determined over time for the plurality of monitoring sessions are consistent (YES branch of 608), the computing device generates one or more reports including an indication of the consistent predicted outcomes for the patient determined over the plurality of monitoring sessions (610).

In some examples, the computing device may compare patient data obtained during a current monitoring session to corresponding baseline(s) established for the patient based on patient data obtained during one or more previous monitoring sessions. In some examples, the computing device may compare one or more maternal and/or fetal outcomes predicted based on data obtained during a current monitoring session to corresponding baseline(s) established for the patient based on one or more maternal and/or fetal outcomes predicted based on data gathered during one or more previous monitoring sessions.

If one or more of the predicted outcomes determined over time for the plurality of monitoring sessions is not consistent (NO branch of 608), the computing device identifies one or more changes in the patient data for the patient over time for the plurality of monitoring sessions (612). The purpose of identifying these changes is to determine whether any of those changes may have resulted in the inconsistency in the predicted outcomes. For example, if a change to fetal heart rate variability (or any other feature(s) or parameter(s)) is detected from a first monitoring session to a second monitoring session, this may account for the change in one or more predicted outcomes from the first monitoring session as compared to the second monitoring session. The computing device generates one or more reports including an indication of the inconsistent predicted outcomes for the patient determined over time for the plurality of monitoring sessions and the one or more changes in the data detected over time for the plurality of monitoring sessions (614). By so doing, the techniques of the disclosure inform the clinicians/providers that changes to the health status of the mother and/or the fetus have occurred, facilitating rapid interventions if necessary, and helping to improve pregnancy outcomes for both mother and fetus.

The process (600) may be repeated each time another monitoring session is performed to continue longitudinal monitoring of the patient.

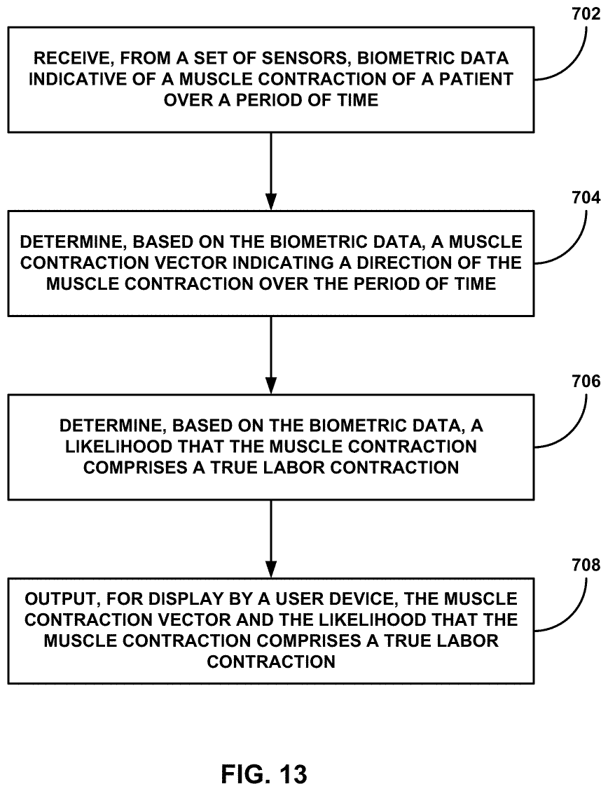

FIG. 13 is a flow diagram illustrating an example operation for generating information corresponding to one or more uterine contractions of a patient, in accordance with one or more techniques of this disclosure. The example operation is described with respect to wearable device 150, computing device 200, and FMS 300 of FIGS. 1-4, and components thereof. However, the techniques of FIG. 13 may be performed by different components of wearable device 150, computing device 200, and FMS 300, or by additional or alternative medical device systems.

FMS 300 may receive, from sensors 152, biometric data indicative of a muscle contraction of patient 120 over a period of time (702). In some examples, biometric data may include a set of electrical potential vector signals that indicate muscle activity of the patient 120. FMS 300 may determine, based on the biometric data, a muscle contraction vector indicating a direction of the muscle contraction over the period of time (704). In some examples, FMS 300 may execute a machine learning model in order to determine the muscle contraction vector indicating the direction of the muscle contraction over the period of time.

FMS 300 may determine, based on the biometric data, a likelihood that the muscle contraction comprises a true labor uterine contraction (706). In some examples, FMS 300 may execute a machine learning model in order to determine the likelihood that the muscle contraction comprises a true labor uterine contraction. FMS 300 may output, for display by a user device, the muscle contraction vector and the likelihood that the muscle contraction comprises a true labor uterine contraction (708). Outputting the muscle contraction vector and the likelihood may assist a clinician in providing healthcare to the patient.

Additional examples of components, devices, apparatus, methods, and/or systems which may be used in connection with one or more aspects of this disclosure are described in U.S. Pat. No. 9,579,055, issued Feb. 28, 2017, U.S. Pat. No. 10,292,652, issued May 21, 2019, and United States Patent Application Publication No. 2020/0113470, published on Apr. 16, 2020, each of which is incorporated herein by reference in its entirety.

In one or more examples, the functions described may be implemented in any combination of processing circuitry, including hardware, software, firmware, or any combination thereof. If implemented in software, the functions may be stored on or transmitted over a computer-readable medium as one or more instructions or code and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable medium.

By way of example, and not limitation, such computer-readable storage media can include RAM, ROM, EEPROM, CD-ROM or other optical disk storage, magnetic disk storage, or other magnetic storage devices, flash memory, or any other medium that can be used to store program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transitory media, but are instead directed to non-transitory, tangible storage media. Disk and disc, as used herein, includes compact disc (CD), laser disc, optical disc, digital versatile disc (DVD), floppy disk and Blu-ray disc, where disks usually reproduce data magnetically, while discs reproduce data optically with lasers. Combinations of the above should also be included within the scope of computer-readable media.

Instructions may be executed by one or more processors, such as one or more DSPs, general purpose microprocessors and/or microcontrollers, ASICs, FPGAs, or other equivalent integrated or discrete logic circuitry, as well as any combination of such components. Accordingly, the term “processor,” as used herein may refer to any of the foregoing structures or any other structure suitable for implementation of the techniques described herein. In addition, in some aspects, the functionality described herein may be provided within dedicated hardware and/or software modules. Also, the techniques could be fully implemented in one or more circuits or logic elements.

The techniques of this disclosure may be implemented in a wide variety of devices or apparatuses, including a wireless communication device, a microprocessor, an integrated circuit (IC) or a set of ICs (e.g., a chip set). Various components, modules, or units are described in this disclosure to emphasize functional aspects of devices configured to perform the disclosed techniques, but do not necessarily require realization by different hardware units. Rather, as described above, various units may be combined in a hardware unit or provided by a collection of interoperative hardware units, including one or more processors as described above, in conjunction with suitable software and/or firmware, and/or any other type or combination of processing circuitry.

Various examples have been described. These and other examples are within the scope of the following claims.

What is claimed is:

1. A system comprising:
a memory; and
one or more processors in communication with the memory, wherein the one or more processors are configured to:
receive, from a set of sensors, biometric data indicative of a muscle contraction of a patient over a period of time;
determine, based on the biometric data, a muscle contraction vector indicating a direction of the muscle contraction over the period of time;
determine, based on the biometric data, a likelihood that the muscle contraction comprises a true labor uterine contraction; and
output, for display by a user device, the muscle contraction vector indicating the direction of the muscle contraction and the likelihood that the muscle contraction comprises a true labor uterine contraction.

2. The system of claim 1, further comprising a wearable device comprising:
a wearable band configured to be worn about a torso of the patient; and
the set of sensors affixed to the wearable band, wherein each sensor of the set of sensors is configured to collect a respective electrical signal of a set of electrical signals, wherein the biometric data comprises the set of electrical signals, and
wherein the set of sensors are configured to output the set of electrical signals to the one or more processors.

3. The system of claim 2, wherein the set of sensors are arranged on the wearable band such that when the wearable band is worn about the torso of the patient, each sensor of the set of sensors is located proximate to a location on the torso of the patient, wherein the set of sensors include a reference sensor, and wherein the one or more processors are configured to:
determine, based on the set of electrical signals, a set of electrical potential vector signals, wherein each electrical potential vector signal of the set of electrical potential vector signals represents a difference between an electrical signal of the set of electrical signals collected by the reference sensor and an electrical signal of the set of electrical signals collected by another sensor of the set of sensors;
determine, based on the set of electrical potential vector signals, the muscle contraction vector; and
determine, based on the set of electrical potential vector signals, the likelihood that the muscle contraction comprises a true labor uterine contraction.

4. The system of claim 3, wherein to determine the muscle contraction vector, the one or more processors are configured to:
process the set of electrical potential vector signals to identify a direction of a movement of the muscle contraction over the period of time relative to the torso of the patient; and
process the set of electrical potential vector signals to identify a magnitude of a strength of the muscle contraction over the period of time.

5. The system of claim 1, wherein the one or more processors are configured to determine the likelihood that the muscle contraction comprises a true labor uterine contraction based on the direction of the muscle contraction over the period of time.

6. The system of claim 1, wherein the memory is configured to store a machine learning model, and wherein the processing circuitry is configured to:
execute the machine learning model to determine the muscle contraction vector indicating the direction of the muscle contraction over the period of time; and
execute the machine learning model to determine the likelihood that the muscle contraction comprises a true labor uterine contraction.

7. The system of claim 6,
wherein the memory is configured to store training data comprising a plurality of biometric training data samples, wherein the plurality of biometric data training samples comprises a first set of biometric data training samples that each indicate a true labor uterine contraction and a second set of biometric data training samples that each indicate a non-labor uterine contraction, and wherein the one or more processors are configured to:
train the machine learning model by identifying one or more patterns associated with the first set of biometric data training samples and identifying one or more patterns associated with the second set of biometric data training samples.

8. The system of claim 7, wherein to execute the machine learning model to determine the likelihood that the muscle contraction comprises a true labor uterine contraction, the one or more processors are configured to:
process the biometric data to identify one or more patterns corresponding to the muscle contraction of the patient over the period of time; and
determine the likelihood that the muscle contraction comprises a true labor uterine contraction based on the one or more patterns corresponding to the muscle contraction, the one or more patterns associated with the first set of biometric data training samples, and the one or more patterns associated with the second set of biometric data training samples.

9. The system of claim 1, wherein the one or more processors are further configured to:
determine, based on the biometric data, a timeseries indicating a strength of the muscle contraction of the patient over the period of time; and
output, for display by the user device, the timeseries indicating the strength of the muscle contraction of the patient over the period of time.

10. The system of claim 1, wherein the one or more processors are further configured to:
receive individual data corresponding to the patient;
determine, based on the biometric data and the individual data corresponding to the patient, the direction of the muscle contraction over the period of time; and
determine, based on the biometric data and the individual data corresponding to the patient, the likelihood that the muscle contraction comprises a true labor uterine contraction.

11. A method comprising:
receiving, by one or more processors from a set of sensors, biometric data indicative of a muscle contraction of a patient over a period of time, wherein the one or more processors are in communication with a memory;
determining, by the one or more processors based on the biometric data, a muscle contraction vector indicating a direction of the muscle contraction over the period of time;
determining, by the one or more processors based on the biometric data, a likelihood that the muscle contraction comprises a true labor uterine contraction; and
outputting, by the one or more processors for display by a user device, the muscle contraction vector indicating the direction of the muscle contraction and the likelihood that the muscle contraction comprises a true labor uterine contraction.

12. The method of claim 11, further comprising outputting, by the set of sensors, the set of electrical signals to the one or more processors, wherein a wearable device comprises:
a wearable band configured to be worn about a torso of the patient; and
the set of sensors affixed to the wearable band, wherein each sensor of the set of sensors is configured to collect a respective electrical signal of a set of electrical signals, wherein the biometric data comprises the set of electrical signals.

13. The method of claim 12,
wherein the set of sensors are arranged on the wearable band such that when the wearable band is worn about the torso of the patient, each sensor of the set of sensors is located proximate to a location on the torso of the patient, wherein the set of sensors include a reference sensor, and wherein the method further comprises:
determining, by the one or more processors based on the set of electrical signals, a set of electrical potential vector signals, wherein each electrical potential vector signal of the set of electrical potential vector signals represents a difference between an electrical signal of the set of electrical signals collected by the reference sensor and an electrical signal of the set of electrical signals collected by another sensor of the set of sensors;
determining, by the one or more processors based on the set of electrical potential vector signals, the muscle contraction vector; and
determining, by the one or more processors based on the set of electrical potential vector signals, the likelihood that the muscle contraction comprises a true labor uterine contraction.

14. The method of claim 13, wherein determining the muscle contraction vector comprises:
processing the set of electrical potential vector signals to identify a direction of a movement of the muscle contraction over the period of time relative to the torso of the patient; and
processing the set of electrical potential vector signals to identify a magnitude of a strength of the muscle contraction over the period of time.

15. The method of claim 11, further comprising determining, by the one or more processors, the likelihood that the muscle contraction comprises a true labor uterine contraction based on the direction of the muscle contraction over the period of time.

16. The method of claim 11, wherein the memory is configured to store a machine learning model, and wherein the method further comprises:
executing, by the one or more processors, the machine learning model to determine the muscle contraction vector indicating the direction of the muscle contraction over the period of time; and
executing, by the one or more processors, the machine learning model to determine the likelihood that the muscle contraction comprises a true labor uterine contraction.

17. The method of claim 16,
wherein the memory is configured to store training data comprising a plurality of biometric training data samples, wherein the plurality of biometric data training samples comprises a first set of biometric data training samples that each indicate a true labor uterine contraction and a second set of biometric data training samples that each indicate a non-labor uterine contraction, and wherein the method further comprises:
training, by the one or more processors, the machine learning model by identifying one or more patterns associated with the first set of biometric data training samples and identifying one or more patterns associated with the second set of biometric data training samples.

18. The method of claim 17, wherein executing the machine learning model to determine the likelihood that the muscle contraction comprises a true labor uterine contraction comprises:
processing the biometric data to identify one or more patterns corresponding to the muscle contraction of the patient over the period of time; and
determining, the likelihood that the muscle contraction comprises a true labor uterine contraction based on the one or more patterns corresponding to the muscle contraction, the one or more patterns associated with the first set of biometric data training samples, and the one or more patterns associated with the second set of biometric data training samples.

19. The method of claim 11, wherein the method further comprises:
determining, by the one or more processors based on the biometric data, a timeseries indicating a strength of the muscle contraction of the patient over the period of time; and
determining, by the one or more processors for display by the user device, the timeseries indicating the strength of the muscle contraction of the patient over the period of time.

20. A computer readable medium comprising instructions that when executed cause one or more processors to:
receive, from a set of sensors, biometric data indicative of a muscle contraction of a patient over a period of time;
determine, based on the biometric data, a muscle contraction vector indicating a direction of the muscle contraction over the period of time;
determine, based on the biometric data, a likelihood that the muscle contraction comprises a true labor uterine contraction; and
output, for display by a user device, the muscle contraction vector indicating the direction of the muscle contraction and the likelihood that the muscle contraction comprises a true labor uterine contraction.
